(19) United States
(12) Patent Application Publication
(10) Pub. No.: US 2024/0180836 A1
(43) Pub. Date: Jun. 6, 2024

(54) Cationic Lipid Compound and Composition for Delivery of Nucleic Acids and Use Thereof

(71) Applicants: Shenzhen Rhegen Biotechnology Co., Ltd., Shenzhen City (CN); Wuhan Rhegen Biotechnology Co.,Ltd., Wuhan City (CN)

(72) Inventors: Yong HU, Shenzhen City (CN); Yafei LI, Shenzhen City (CN); Zhaoyu HU, Shenzhen City (CN)

(21) Appl. No.: 18/410,875

(22) Filed: Jan. 11, 2024

Related U.S. Application Data
(63) Continuation of application No. PCT/CN2023/110927, filed on Aug. 3, 2023.

(30) Foreign Application Priority Data
Aug. 3, 2022 (CN) 202210926949.9

(51) Int. Cl. A61K 9/127; A61K 38/18; A61K 48/00; C07C 327/22; C07C 327/30

(52) U.S. Cl. CPC A61K 9/1272; A61K 38/1816; A61K 48/0033; C07C 327/22; C07C 327/30

(57) ABSTRACT

The cationic lipid compound and composition for delivering nucleic acids as shown in formula (I) and their use are disclosed. The use of nano lipid particles with the compound as a key component in nucleic acid delivery is also disclosed, including the composition, preparation method, and usage method of the delivery carrier.

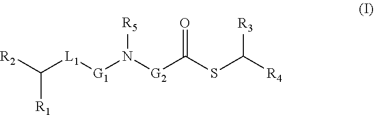

### FIELD OF TECHNOLOGY

The present disclosure relates to the field of lipid delivery carriers. Particularly, The present disclosure relates to a class of cationic lipid compounds which, when combined with other lipid components, are capable of forming drug-carrying nano-lipid particles, thereby enabling the delivery of nucleic acids from outside to inside of cells in vitro and in vivo. Specifically, the present disclosure relates to a cationic lipid compound and a composition for delivery of nucleic acids and use thereof.

### BACKGROUND ART

Nucleic acid drugs replace, compensate, block or modify specific genes by introducing exogenous genes into target cells or tissues to achieve the purpose of treating and preventing diseases. They are relatively simpler to develop and produce, and have the advantages of short R&D cycle, high success rate in clinical development, and better plasticity for improvement. Nucleic acid vaccines, as one of the mainstays of COVID-19 prevention in recent years, have also proved their great potential in the market.

However, naked mRNA has a short circulation time in vivo, is easily degraded, and is difficult to enter target cells or target tissues. Therefore, improving the in vivo delivery efficiency of mRNA drugs is one of the key directions to improve the effectiveness of this class of products.

Currently, the most widely used delivery carriers for nucleic acid drugs are lipid nanoparticles, which have the characteristics of improving the efficacy of gene drugs as well as targeted delivery effects, and can protect nucleic acids from rapid degradation in vivo, so as to prolong the circulation time and enhance targeted delivery. The lipid nanoparticles are comprised of 2-4 lipid components, including cationic lipid compounds, 0-2 auxiliary lipids and 0-1 PEG lipids, in which cationic lipid compounds play a key role in encapsulation and release of nucleic acids. Therefore, it is crucial to develop novel, efficient and low-toxic cationic lipid compounds.

### SUMMARY OF THE DISCLOSURE

The present disclosure provides a class of sulfur-containing cationic lipid compounds that are readily degradable and rapidly metabolized in vivo, including pharmaceutically acceptable salts thereof and stereoisomers or tautomers thereof. They are primarily used in combination with other lipid components in specific ratios, to form lipid nanoparticles for delivery of prophylactic or therapeutic agents, such as therapeutic nucleic acids.

Another object of the present disclosure is to provide methods for synthesizing this class of lipid compounds, by using readily available raw materials via a reaction route with mild conditions, high product yields, low instrumentation requirements and simplicity of operation.

In some examples, the therapeutic nucleic acids include plasmid DNA, messenger RNA, antisense oligonucleotides (ASON), micro RNA (miRNA), interfering RNA (micRNA), dicer substrate RNA, complementary DNA (cDNA).

The present disclosure also provides formulations and methods of use of such cationic lipid compounds when used in combination with other lipid components, and applications thereof in cellular and animal models.

In embodiments of the present disclosure, there is provided a cationic lipid compound having the following structure of formula (I):

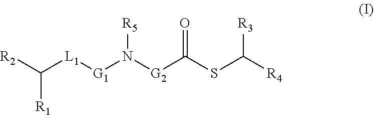

or a pharmaceutically acceptable salt, tautomer or stereoisomer thereof,
wherein:
L₁ is any one of —C(═O)—, —OC(═O)—, —C(═O)O—, —OC(═O)O—, —O—, —S—, —S—S—, —C(═O)S—,
—SC(═O)—, —N(R₆)C(═O)—, —C(═O)N(R₆)—, —N(R₆)C(═O)O—, —OC(═O)N(R₆)—, —SC(═O)N(R₆)—, —N(R₆)C(═O)S—, —C(═S)—, —SC(═S)— and —C(═S)S—, and R₆ is H or a C₁-C₁₂ alkyl;
G₁ and G₂ are each independently a C₃-C₁₀ alkylene;
R₁ is a C₂-C₁₂ alkyl;
R₂ is H or a C₂-C₁₂ alkyl;
R₃ is a C₂-C₁₂ alkyl;
R₄ is H or a C₂-C₁₂ alkyl;
R₅ is H, a C₁-C₆ alkyl, —R₇—OH, —R₇—OC(═O)CH₃, —R₇—NHC(═O)—CH₃, —R₇—OCH₃ or —R₇—NR₈(R₉);
R₇ is a C₂-C₁₈ alkylene;
R₈ and R₉ are C₁-C₈ straight chain alkyl groups, or R₈, R₉ and the N atoms attached thereto form a C₃-C₁₀ heterocyclic group.

In some specific embodiments of the present disclosure, L₁ is —OC(═O)—, —C(═O)O—, —SC(═O)— or —C(═O)S—.

In some specific embodiments of the present disclosure, G₁ and G₂ are C₃-C₈ alkylene.

In some specific embodiments of the present disclosure, R₇ is a C₂-C₆ alkylene.

In some specific embodiments of the present disclosure, R₁ and R₃ are each independently a C₃-C₉ alkyl; and R₂ and R₄ are H or C₃-C₉ alkyl.

In some specific embodiments of the present disclosure, one and only one of R₂ and R₄ is H.

In some specific embodiments of the present disclosure, R₅ is —R₇—OH, and R₇ is a C₂-C₈ alkylene.

In some specific embodiments of the present disclosure, R₅ is —R₇—N(CH₂CH₃)CH₂CH₃.

In some specific embodiments of the present disclosure, the structure of —C(R₁)R₂ or —C(R₃)R₄ in the structure of formula (I) each independently conforms to the following characteristics:

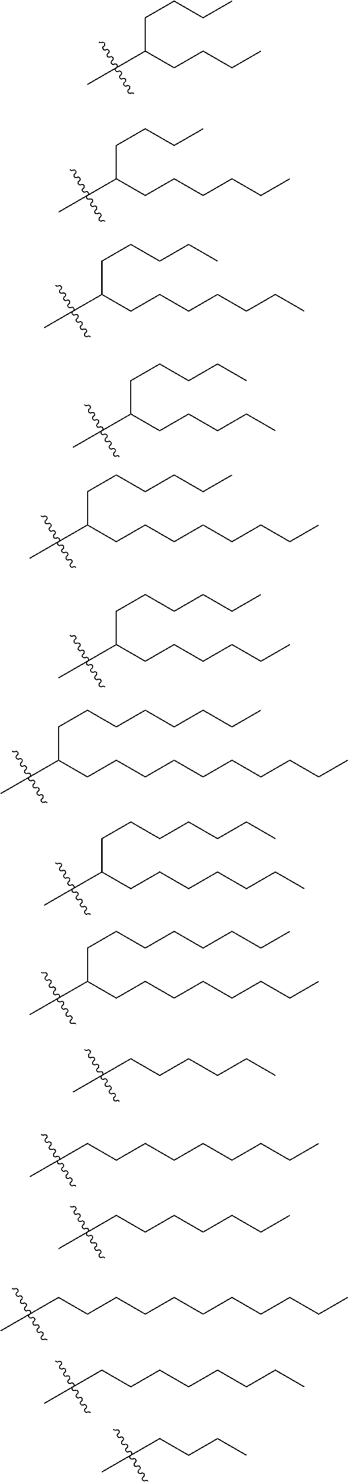

According to some specific embodiments of the present disclosure, wherein:
L₁ is —OC(═O)— or —SC(═O)—;
G₁ is a C₅-C₈alkylene;
G₂ is a C₄-C₈ alkylene;
the structure of —C(R₁)R₂ is selected from:

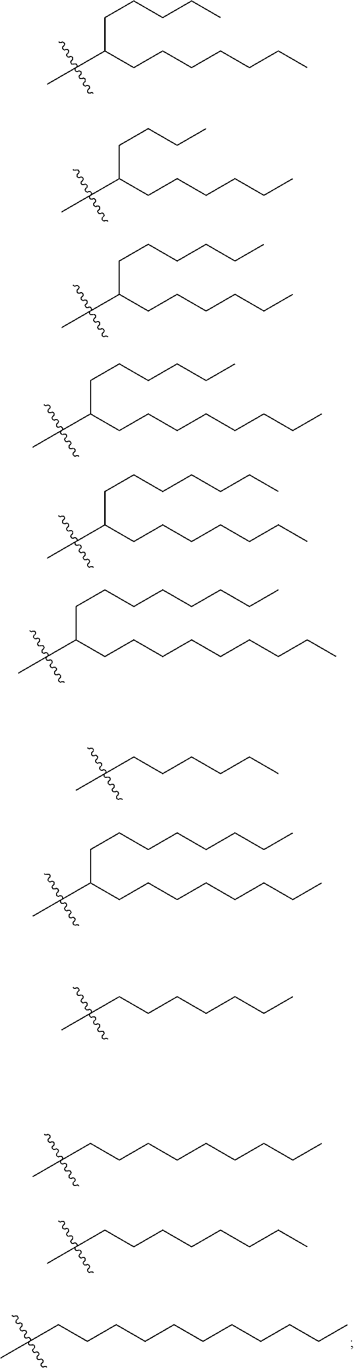

the structure of —C(R₃)R₄ is selected from:

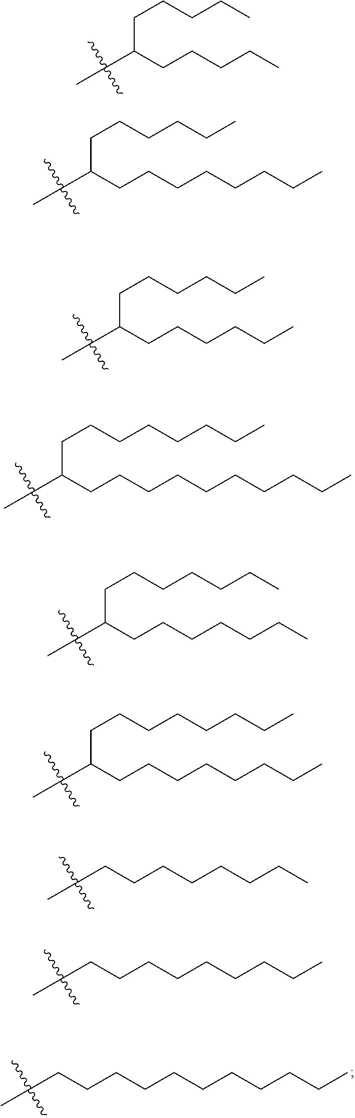

R₅ is —R₇—OH or —R₇—NR₈(R₉);
R₇ is a C₂-C₆ alkylene;
R₈ and R₉ are C₁-C₈ straight chain alkyl groups, or R₈, R₉ and the N atoms attached thereto form a C₃-C₁₀ heterocyclic alkyl group.

In some specific embodiments of the present disclosure, the cationic lipid compound has one of the structures shown in the following table:

| No. | Structure |
|---|---|
| 1 | 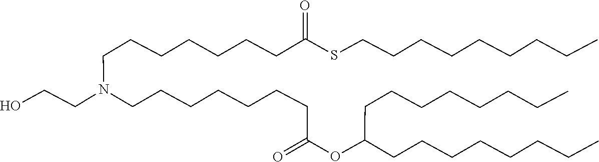 [SMILES: CCCCCCCCCSC(=O)CCCCCCCN(CCO)CCCCCCCC(=O)OC(CCCCCCCC)CCCCCCCC] |
| 2 | 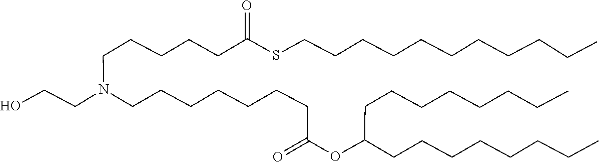 [SMILES: CCCCCCCCCCCSC(=O)CCCCCN(CCO)CCCCCCCC(=O)OC(CCCCCCCC)CCCCCCCC] |
| 3 | 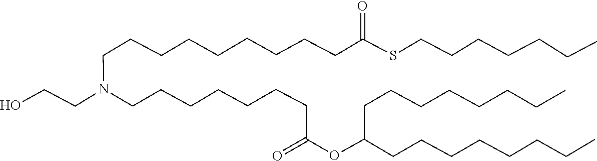 [SMILES: CCCCCCCCC(CCCCCCCC)OC(=O)CCCCCCCN(CCO)CCCCCCCCCC(=O)SCCCCCCC] |
| 4 | 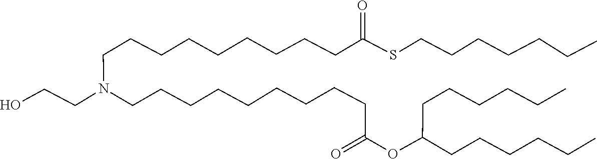 [SMILES: CCCCCCCSC(=O)CCCCCCCCCN(CCO)CCCCCCCCCC(=O)OC(CCCCCC)CCCCCC] |
| 5 | 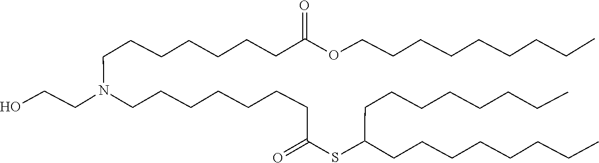 [SMILES: CCCCCCCCCOC(=O)CCCCCCCN(CCO)CCCCCCCC(=O)SC(CCCCCCCC)CCCCCCCC] |
| 6 | 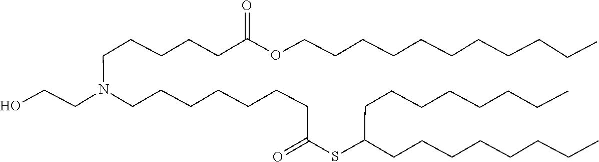 [SMILES: CCCCCCCCCCCOC(=O)CCCCCN(CCO)CCCCCCCC(=O)SC(CCCCCCCC)CCCCCCCC] |
| 7 | 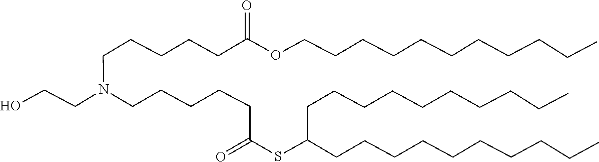 [SMILES: CCCCCCCCCCCOC(=O)CCCCCN(CCO)CCCCCC(=O)SC(CCCCCCCCCC)CCCCCCCCCC] |
| 8 | 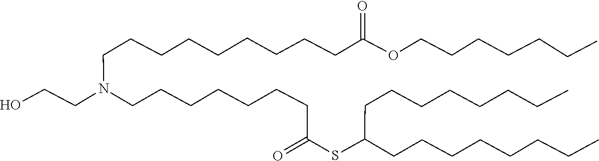 [SMILES: CCCCCCCCC(CCCCCCCC)SC(=O)CCCCCCCN(CCO)CCCCCCCCCC(=O)OCCCCCCC] |
| 9 | 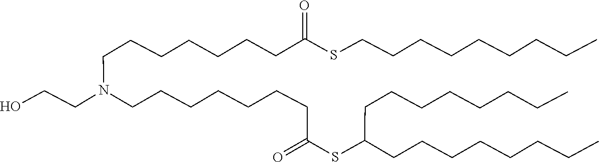 [SMILES: CCCCCCCCCSC(=O)CCCCCCCN(CCO)CCCCCCCC(=O)SC(CCCCCCCC)CCCCCCCC] |
| 10 | 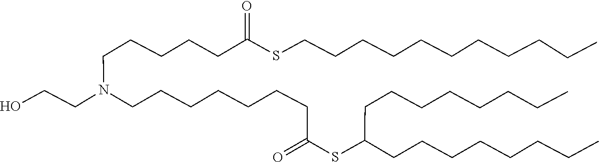 [SMILES: CCCCCCCCCCCSC(=O)CCCCCN(CCO)CCCCCCCC(=O)SC(CCCCCCCC)CCCCCCCC] |
| 11 | 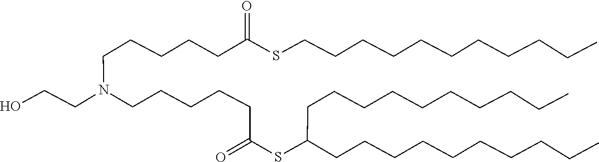 [SMILES: CCCCCCCCCCCSC(=O)CCCCCN(CCO)CCCCCC(=O)SC(CCCCCCCCCC)CCCCCCCCCC] |
| 12 | 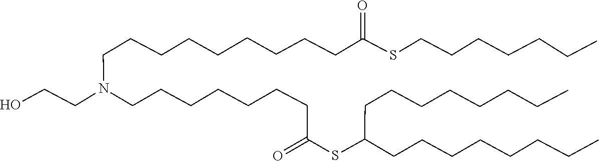 [SMILES: CCCCCCCCC(CCCCCCCC)SC(=O)CCCCCCCN(CCO)CCCCCCCCCC(=O)SCCCCCCC] |
| 13 | 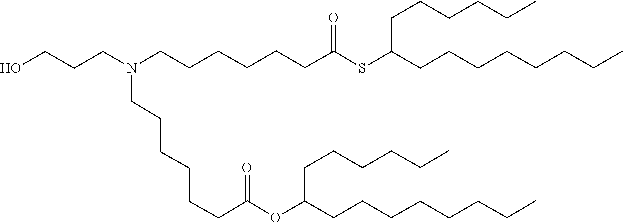 [SMILES: CCCCCCCCC(CCCCCC)OC(=O)CCCCCCN(CCCO)CCCCCCC(=O)SC(CCCCCC)CCCCCCCC] |
| 14 | 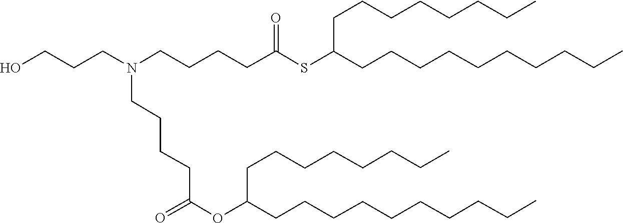 [SMILES: CCCCCCCCCCC(CCCCCCCC)OC(=O)CCCCN(CCCO)CCCCC(=O)SC(CCCCCCCC)CCCCCCCCCC] |
| 15 | 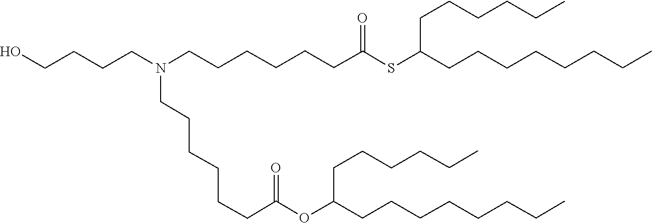 [SMILES: CCCCCCCCC(CCCCCC)OC(=O)CCCCCCN(CCCCO)CCCCCCC(=O)SC(CCCCCC)CCCCCCCC] |
| 16 | 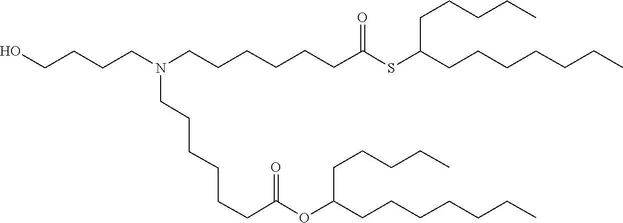 [SMILES: CCCCCCCC(CCCCC)OC(=O)CCCCCCN(CCCCO)CCCCCCC(=O)SC(CCCCC)CCCCCCC] |
| 17 | 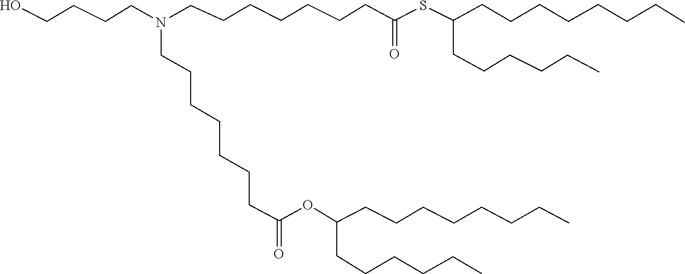 [SMILES: CCCCCCCCC(CCCCCC)OC(=O)CCCCCCCN(CCCCO)CCCCCCCC(=O)SC(CCCCCC)CCCCCCCC] |
| 18 | 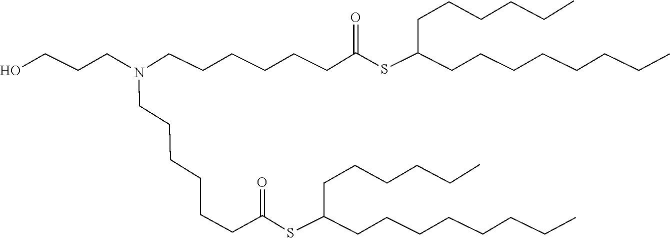 [SMILES: CCCCCCCCC(CCCCCC)SC(=O)CCCCCCN(CCCO)CCCCCCC(=O)SC(CCCCCC)CCCCCCCC] |
| 19 | 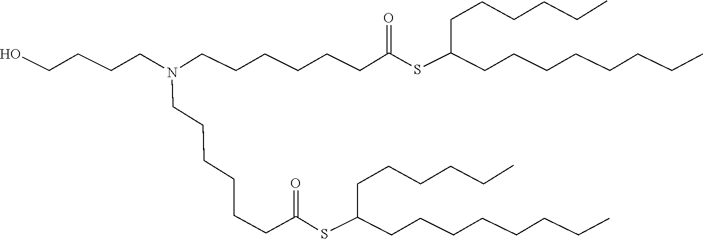 [SMILES: CCCCCCCCC(CCCCCC)SC(=O)CCCCCCN(CCCCO)CCCCCCC(=O)SC(CCCCCC)CCCCCCCC] |
| 20 | 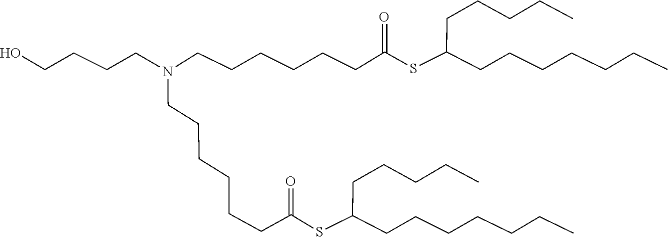 [SMILES: CCCCCCCC(CCCCC)SC(=O)CCCCCCN(CCCCO)CCCCCCC(=O)SC(CCCCC)CCCCCCC] |
| 21 | 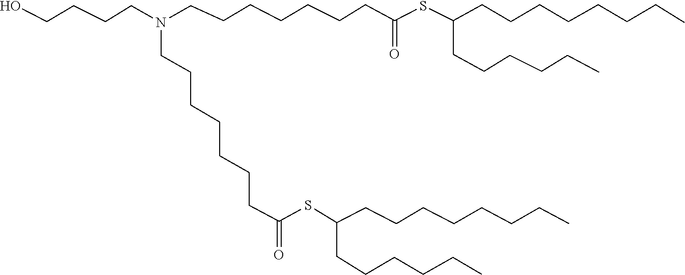 [SMILES: CCCCCCCCC(CCCCCC)SC(=O)CCCCCCCN(CCCCO)CCCCCCCC(=O)SC(CCCCCC)CCCCCCCC] |
| 22 | 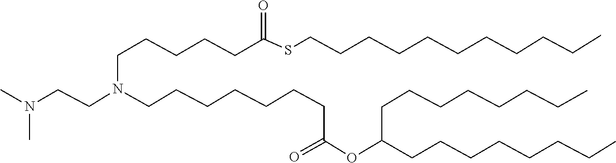 [SMILES: CCCCCCCCCCCSC(=O)CCCCCN(CCCCCCCC(=O)OC(CCCCCCCC)CCCCCCCC)CCN(C)C] |
| 23 | 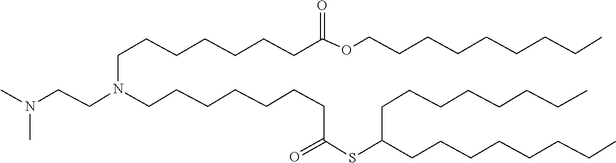 [SMILES: CCCCCCCCCOC(=O)CCCCCCCN(CCCCCCCC(=O)SC(CCCCCCCC)CCCCCCCC)CCN(C)C] |
| 24 | 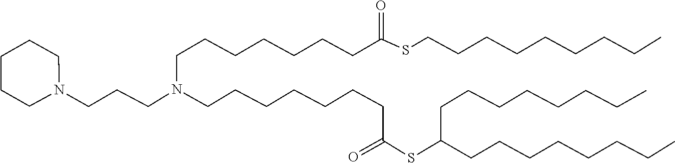 [SMILES: CCCCCCCCCSC(=O)CCCCCCCN(CCCCCCCC(=O)SC(CCCCCCCC)CCCCCCCC)CCCN1CCCCC1] |
| 25 | 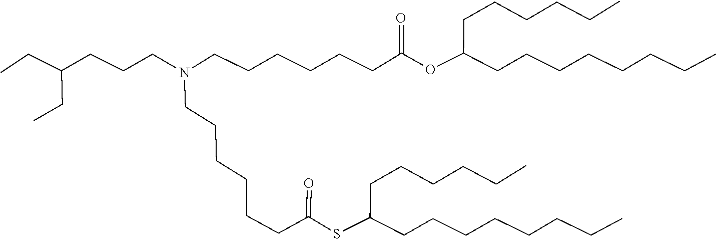 [SMILES: CCCCCCCCC(CCCCCC)OC(=O)CCCCCCN(CCCCCCC(=O)SC(CCCCCC)CCCCCCCC)CCCC(CC)CC] |
| 26 | 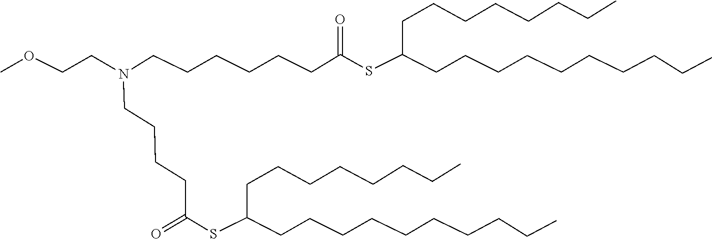 [SMILES: CCCCCCCCCCC(CCCCCCCC)SC(=O)CCCCCCN(CCCCC(=O)SC(CCCCCCCC)CCCCCCCCCC)CCOC] |
| 27 | 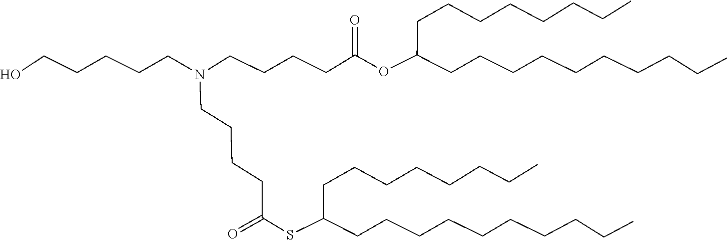 [SMILES: CCCCCCCCCCC(CCCCCCCC)OC(=O)CCCCN(CCCCCO)CCCCC(=O)SC(CCCCCCCC)CCCCCCCCCC] |
| 28 | 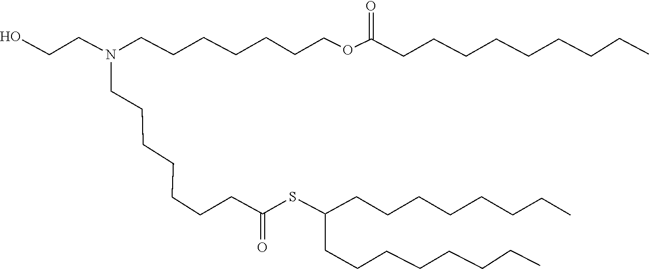 [SMILES: CCCCCCCCCC(=O)OCCCCCCCN(CCO)CCCCCCCC(=O)SC(CCCCCCCC)CCCCCCCC] |
| 29 | 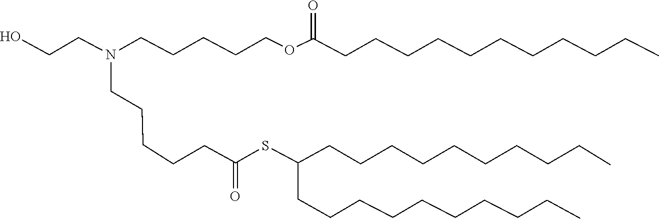 [SMILES: CCCCCCCCCCCC(=O)OCCCCCN(CCO)CCCCCC(=O)SC(CCCCCCCCCC)CCCCCCCCCC] |
| 30 | 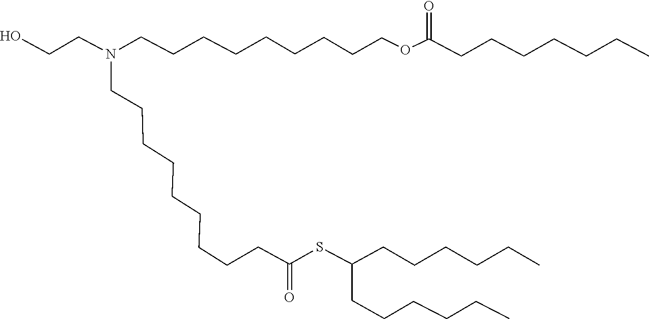 [SMILES: CCCCCCCC(=O)OCCCCCCCCCN(CCO)CCCCCCCCCC(=O)SC(CCCCCC)CCCCCC] |
| 31 | 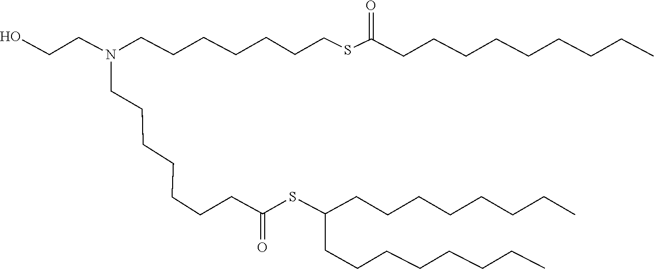 [SMILES: CCCCCCCCCC(=O)SCCCCCCCN(CCO)CCCCCCCC(=O)SC(CCCCCCCC)CCCCCCCC] |
| 32 | 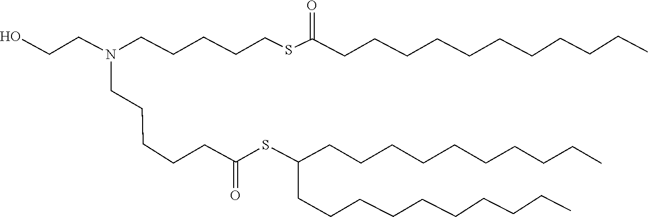 [SMILES: CCCCCCCCCCCC(=O)SCCCCCN(CCO)CCCCCC(=O)SC(CCCCCCCCCC)CCCCCCCCCC] |
| 33 | 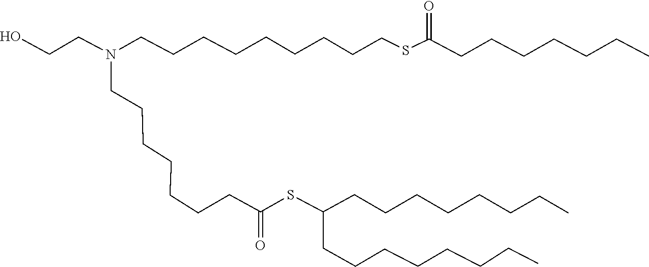 [SMILES: CCCCCCCCC(CCCCCCCC)SC(=O)CCCCCCCN(CCO)CCCCCCCCCSC(=O)CCCCCCC] |
| 34 | 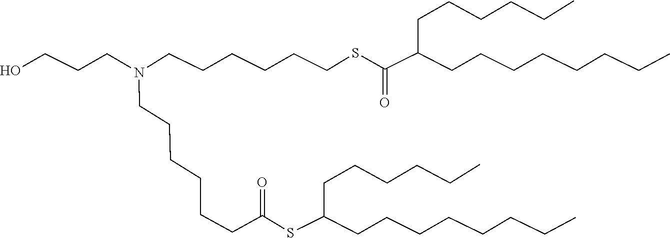 [SMILES: CCCCCCCCC(CCCCCC)SC(=O)CCCCCCN(CCCO)CCCCCCSC(=O)C(CCCCCC)CCCCCCCC] |
| 35 | 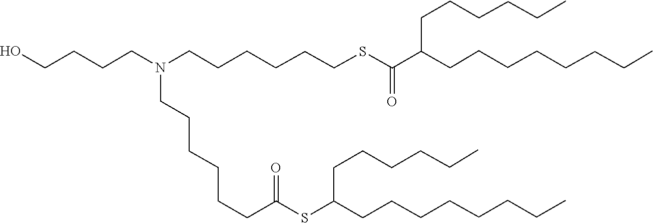 [SMILES: CCCCCCCCC(CCCCCC)SC(=O)CCCCCCN(CCCCO)CCCCCCSC(=O)C(CCCCCC)CCCCCCCC] |
| 36 | 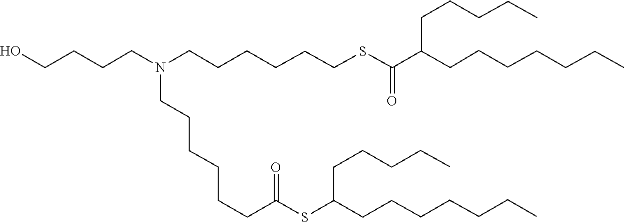 [SMILES: CCCCCCCC(CCCCC)SC(=O)CCCCCCN(CCCCO)CCCCCCSC(=O)C(CCCCC)CCCCCCC] |
| 37 | 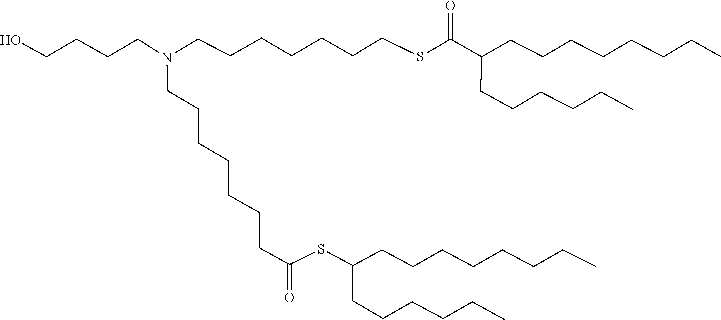 [SMILES: CCCCCCCCC(CCCCCC)SC(=O)CCCCCCCN(CCCCO)CCCCCCCSC(=O)C(CCCCCC)CCCCCCCC] |
| 38 | 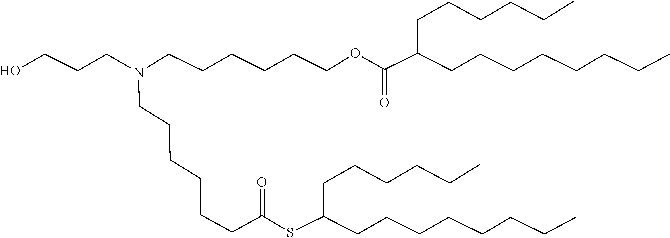 [SMILES: CCCCCCCCC(CCCCCC)SC(=O)CCCCCCN(CCCO)CCCCCCOC(=O)C(CCCCCC)CCCCCCCC] |
| 39 | 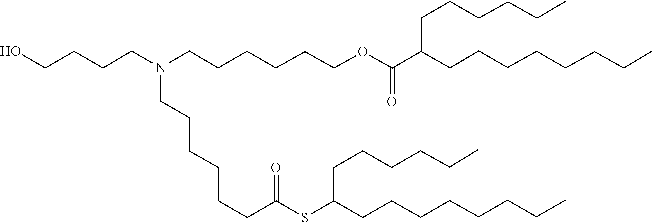 [SMILES: CCCCCCCCC(CCCCCC)SC(=O)CCCCCCN(CCCCO)CCCCCCOC(=O)C(CCCCCC)CCCCCCCC] |
| 40 | 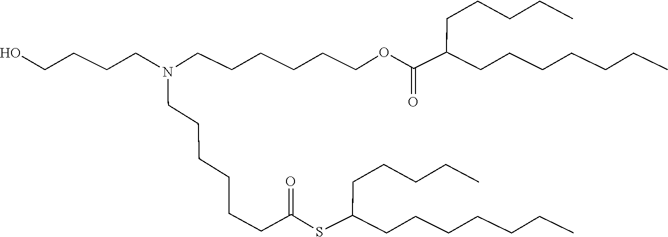 [SMILES: CCCCCCCC(CCCCC)SC(=O)CCCCCCN(CCCCO)CCCCCCOC(=O)C(CCCCC)CCCCCCC] |
| 41 | 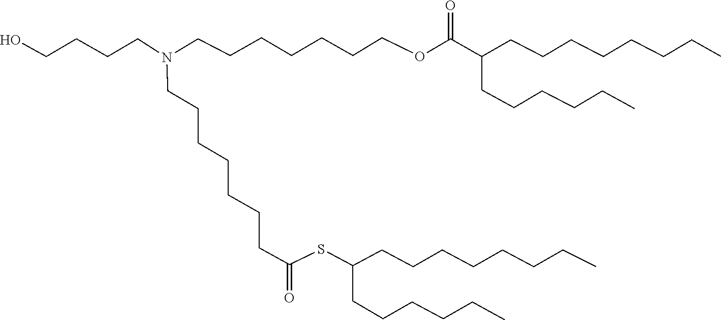 [SMILES: CCCCCCCCC(CCCCCC)SC(=O)CCCCCCCN(CCCCO)CCCCCCCOC(=O)C(CCCCCC)CCCCCCCC] |
| 42 | 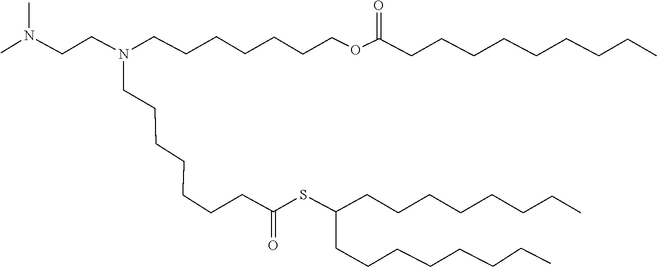 [SMILES: CCCCCCCCCC(=O)OCCCCCCCN(CCCCCCCC(=O)SC(CCCCCCCC)CCCCCCCC)CCN(C)C] |
| 43 | 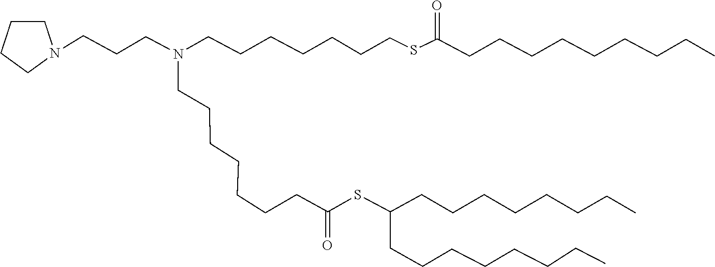 [SMILES: CCCCCCCCCC(=O)SCCCCCCCN(CCCCCCCC(=O)SC(CCCCCCCC)CCCCCCCC)CCCN1CCCC1] |
| 44 | 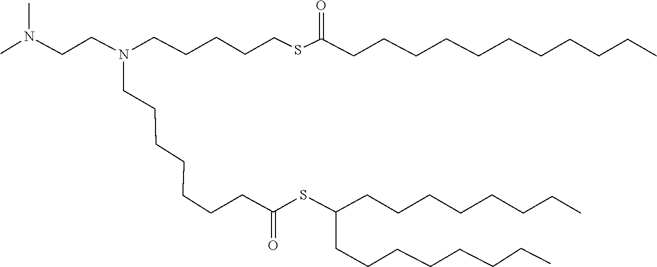 [SMILES: CCCCCCCCCCCC(=O)SCCCCCN(CCCCCCCC(=O)SC(CCCCCCCC)CCCCCCCC)CCN(C)C] |
| 45 | 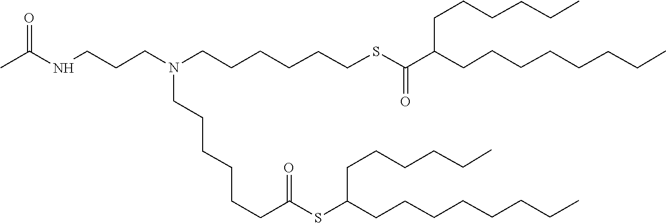 [SMILES: CCCCCCCCC(CCCCCC)SC(=O)CCCCCCN(CCCCCCSC(=O)C(CCCCCC)CCCCCCCC)CCCNC(C)=O] |
| 46 | 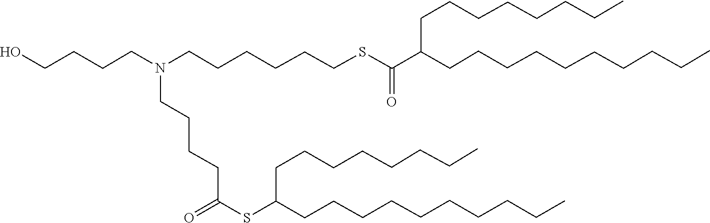 [SMILES: CCCCCCCCCCC(CCCCCCCC)SC(=O)CCCCN(CCCCO)CCCCCCSC(=O)C(CCCCCCCC)CCCCCCCCCC] |

The present disclosure also provides a liposomal formulation comprising one or more cationic lipid compounds of the present disclosure and prophylactic or therapeutic nucleic acids, wherein the liposomal formulation is used for the prevention or treatment of certain diseases.

The liposomal formulation comprises one or more components selected from neutral lipids, charged lipids, steroids and polymer-conjugated lipids. The therapeutic agents used in the present disclosure are therapeutic nucleic acids comprising plasmid DNA, messenger RNA, antisense oligonucleotide (ASON), micro RNA (miRNA), interfering RNA (micRNA), dicer substrate RNA, complementary DNA (cDNA), preferably plasmid DNA, messenger RNA and antisense oligonucleotides.

In some specific embodiments of the present disclosure, the molar ratio of the nucleic acid to the cationic lipid compound is from 20:1 to 1:1.

In some specific embodiments of the present disclosure, the molar ratio of the nucleic acid to the cationic lipid compound is from 10:1 to 4:1.

In some specific embodiments of the present disclosure, the liposomal formulation has a diameter of 50 nm to 300 nm.

In some specific embodiments of the present disclosure, the liposomal formulation has a diameter of 50 nm to 150 nm, or 150 nm to 200 nm.

In some specific embodiments of the present disclosure, it further comprises one or more other lipid components, including, but not limited to, a structural lipid, a steroid and a polymer-conjugated lipid.

In some specific embodiments of the present disclosure, the included steroid is cholesterol.

In some specific embodiments of the present disclosure, the molar ratio of the cholesterol to the cationic lipid compound is from 0:1 to 1.5:1.

In some specific embodiments of the present disclosure, the molar ratio of the cholesterol to the cationic lipid compound is from 0.2:1 to 1.2:1.

In some specific embodiments of the present disclosure, the polymer in the polymer-conjugated lipid is polyethylene glycol (PEG).

In some specific embodiments of the present disclosure, the molar ratio of the cationic lipid compound to the polyethylene glycol-conjugated lipid is from 100:1 to 20:1.

In some specific embodiments of the present disclosure, wherein the polyethylene glycol-conjugated lipid is PEG-DAG, PEG-PE, PEG-SDAG, PEG-cer, PEG-DMG or ALC-0159.

In some specific embodiments of the present disclosure, the liposomal formulation comprises one or more structural lipids selected from DPPG, DSPC, DPPC, DMPC, DOPC, POPC, DOPE and DSPE.

In some specific embodiments of the present disclosure, the structural lipid is DSPC or DOPE.

In some specific embodiments of the present disclosure, the molar ratio of the structural lipid to the cationic lipid compound is from 0:1 to 0.5:1.

In some specific embodiments of the present disclosure, the molar ratio of the structural lipid to the cationic lipid compound is from 0:1 to 0.3:1.

In some specific embodiments of the present disclosure, the liposomal formulation comprises a nucleic acid.

In some specific embodiments of the present disclosure, the nucleic acid is selected from antisense RNA and/or messenger RNA.

In some specific embodiments of the present disclosure, the nucleic acid is messenger RNA.

The present disclosure also provides use of the cationic lipid compound or the liposomal formulation described herein in the preparation of a medicament for inducing protein expression in a subject.

In some specific embodiments of the present disclosure, the subject is a mammal.

In some specific embodiments of the present disclosure, the subject is a non-human primate.

In some specific embodiments of the present disclosure, the subject is a human.

In summary, the present disclosure provides a cationic lipid compound and liposomal formulation for delivery of nucleic acids and use thereof. The technical solution of the present disclosure has the following advantages:

The cationic lipid compound of the present disclosure has a thioester bond. The introduction of the thioester bond makes the compound more easily degradable and improves the rate of in vivo scavenging rate of the lipid compounds, resulting in lower toxicity and fewer residues in vivo of the carriers comprising the compounds. Moreover, the method of preparing the lipid compounds described herein has the advantages of easy availability of raw materials, mild reaction conditions, high product yield, low requirements for instrumentation and simple operation.

### BRIEF DESCRIPTION OF DRAWINGS

FIG. 1 is a graph showing the fluorescence brightness of the compound in Example 14;

FIG. 2 is a graph showing the packed cell volume in Example 15;

FIG. 3 is a graph showing the metabolism rate of cationic lipid in Example 16.

### DETAILED DESCRIPTION OF PREFERRED EMBODIMENTS

The technical solutions of the present disclosure are described in detail below in conjunction with the accompanying drawings and examples, but the protection scope of the present disclosure includes, but is not limited to, them.

### Example 1

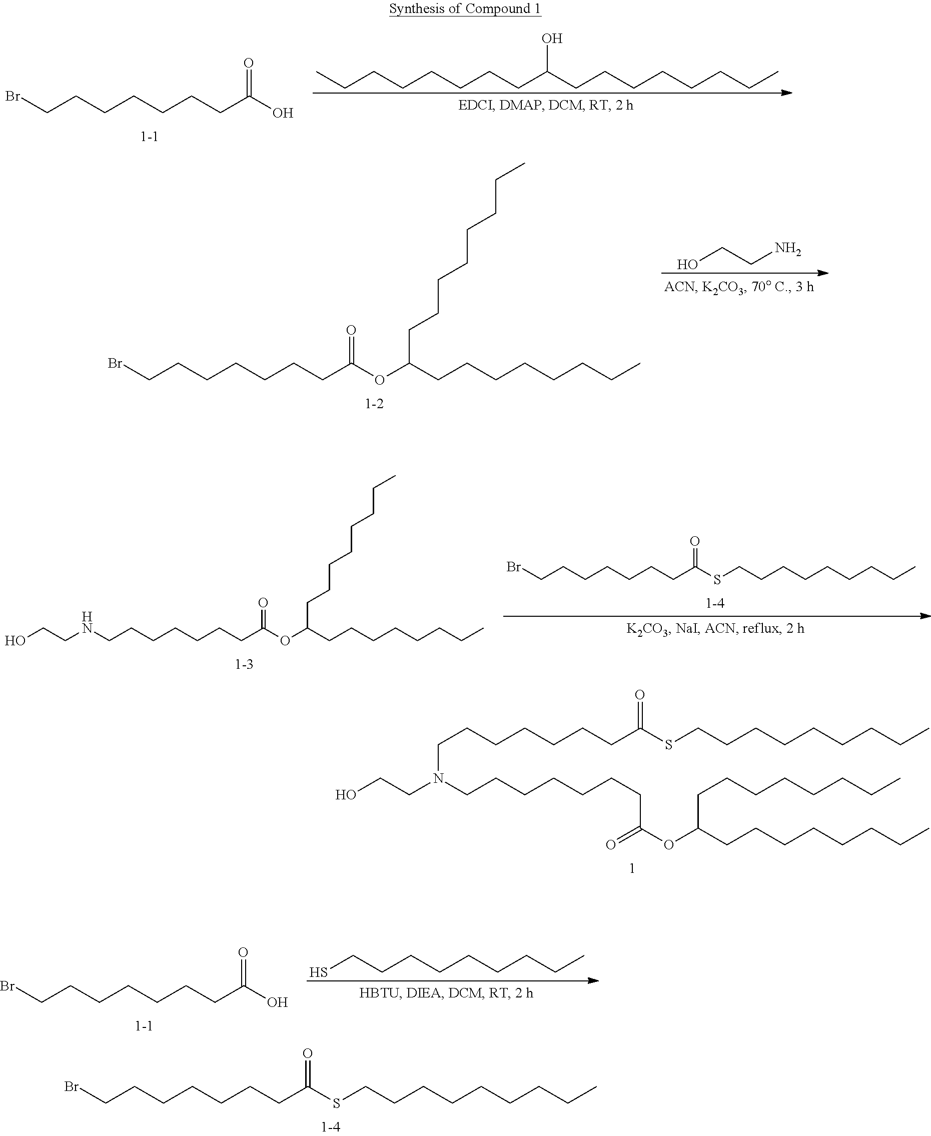

Step 1:

Compound 1-1 (3.0 g) was dissolved in DCM (10 ml) and stirred at room temperature. 1-ethyl-(3-dimethylaminopropyl)carbodiimide hydrochloride (EDCI, 3.35 g), 4-dimethylaminopyridine (DMAP, 1.64 g) and 9-heptadecanol (3.79 g) were weighed sequentially, and added to the reaction system in batches and stirred at room temperature for 2 h. A small amount of the reaction solution was diluted and spotted on the plate in control with a 1-1 standard sample (PE/EA=10/1, phosphomolybdic acid). New spots with reduced polarity were observed. The reaction solution was evaporated under reduced pressure. It was mixed with appropriate amount of silica gel and DCM, and purified (10 g normal phase column, PE/EA, 0-0% 5 min, 0-5% 20 min, 5-5% 5 min, a flow rate of 15 ml/min). The spot plate was monitored, and fractions of the pure product were evaporated to give a colorless oily liquid compound 1-2 (5.5 g, 89% yield).

Step 2:

To a solution of compound 1-2 (5.0 g) and ethanolamine (1.0 g) in acetonitrile (100 mL), potassium carbonate (4.5 g) was added. The mixture was stirred at 70° C. for 2 h. TLC showed complete disappearance of compound 1-2 and generation of a point with increased polarity. The reaction solution was filtered. The resulting filtrate was concentrated to obtain a crude product, which was then was mixed with appropriate amount of silica gel and DCM, and purified (25 g normal phase column, PE/EA, 0-0% 5 min, 0-10% 20 min, 10-10% 5 min, a flow rate of 20 ml/min), to give a colorless oily liquid compound 1-3 (3.0 g, 62.6% yield).

Step 3:

Compound 1-1 (3.0 g) was dissolved in DCM (40 ml) and stirred at room temperature. Benzotriazole-N,N,N′,N′-tetramethyluronium hexafluorophosphate (HBTU, 4.1 g), N,N-diisopropylethylamine (DIEA, 2.3 g) and 1-nonanethiol (1.6 g) were weighed sequentially, and added to the reaction system in batches and stirred at room temperature for 2 h. A small amount of the reaction solution was diluted and spotted on the plate in control with a 1-1 standard sample (PE/EA=10/1, phosphomolybdic acid). New spots with reduced polarity were observed. The reaction solution was evaporated under reduced pressure. It was mixed with appropriate amount of silica gel and DCM, and purified (25 g normal phase column, PE/EA, 0-0% 5 min, 0-5% 20 min, 5-5% 5 min, a flow rate of 20 ml/min), to give a colorless oily liquid compound 1-4 (2.9 g, 89% yield).

Step 4:

Compound 1-3 (500 mg) was dissolved in acetonitrile (10 ml) and stirred at room temperature. Subsequently, NaI (170 mg), K₂CO₃ (470 mg) and compound 1-4 (500 mg) were weighed sequentially, and added to the reaction system in batches and heated and stirred at 85° C. under reflux for 2 h. A small amount of the reaction solution was diluted and spotted on the plate (DCM/MeOH=10/1, Id aqueous ammonia, phosphomolybdic acid). New spots with less polarity than that of 1-3 were observed. The reaction solution was cooled to room temperature and then evaporated under reduced pressure. It was mixed with appropriate amount of silica gel and DCM, and purified (25 g normal phase column, DCM/MeOH, 0.1% aqueous ammonia, 0-0% 10 min, 0-7.5% 20 min, 7.5-7.5% 5 min, a flow rate of 25 ml/min), and concentrated to give a light yellow oily liquid compound 1 (600 mg, 73% yield).

¹H NMR (400 MHz, Chloroform-d) δ 4.01-3.96 (m, 1H), 2.85 (d, J=7.6 Hz, 2H), 2.81-2.75 (m, 2H), 2.79-2.58 (m, 6H), 2.57-2.40 (m, 2H), 2.31-2.27 (m, 2H), 1.95-1.48 (m, 16H), 1.25 (s, 46H), 0.86 (d, J=7.2 Hz, 9H).

### Example 2

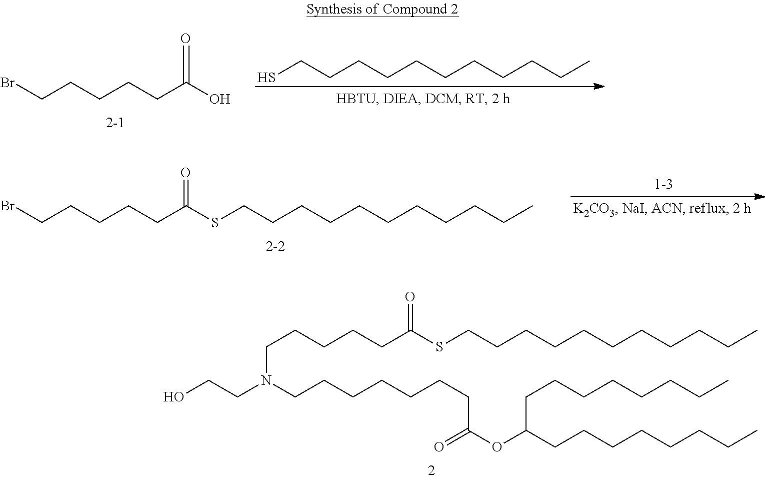 [SMILES: O=C(O)CCCCCBr ; CCCCCCCCCCCS ; CCCCCCCCCCCSC(=O)CCCCCBr ; CCCCCCCCCCCSC(=O)CCCCCN(CCO)CCCCCCCC(=O)OC(CCCCCCCC)CCCCCCCC]

Step 1:

Compound 2-1 (3.0 g) was dissolved in DCM (40 ml) and stirred at room temperature. Benzotriazole-N,N,N′,N′-tetramethyluronium hexafluorophosphate (HBTU, 7.0 g), N,N-diisopropylethylamine (DIEA, 4.0 g) and 1-undecanethiol (3.5 g) were weighed sequentially, and added to the reaction system in batches and stirred at room temperature for 2 h. A small amount of the reaction solution was diluted and spotted on the plate in control with a 2-1 standard sample (PE/EA=10/1, phosphomolybdic acid). New spots with reduced polarity were observed. The reaction solution was evaporated under reduced pressure. It was mixed with appropriate amount of silica gel and DCM, and purified (40 g normal phase column, PE/EA, 0-0% 5 min, 0-5% 20 min, 5-5% 5 min, a flow rate of 30 ml/min), to give a colorless oily liquid compound 2-2 (5.5 g, 89% yield).

Step 2:

Compound 1-3 (500 mg) was dissolved in acetonitrile (10 ml) and stirred at room temperature. Subsequently, NaI (170 mg), K₂CO₃ (470 mg) and compound 2-2 (500 mg) were weighed sequentially, and added to the reaction system in batches and heated and stirred at 85° C. under reflux for 2 h. A small amount of the reaction solution was diluted and spotted on the plate (DCM/MeOH=10/1, Id aqueous ammonia, phosphomolybdic acid). New spots with less polarity than that of 1-3 were observed. The reaction solution was cooled to room temperature and then evaporated under reduced pressure. It was mixed with appropriate amount of silica gel and DCM, purified (25 g normal phase column, DCM/MeOH, 0.1% aqueous ammonia, 0-0% 10 min, 0-7.5% 20 min, 7.5-7.5% 5 min, a flow rate of 25 ml/min), and concentrated to give a light yellow oily liquid compound 2 (600 mg, 73% yield).

¹H NMR (400 MHz, Chloroform-d) δ 4.01-3.96 (m, 1H), 2.92-2.88 (m, 2H), 2.81-2.75 (m, 2H), 2.79-2.58 (m, 6H), 2.57-2.40 (m, 2H), 2.31-2.27 (m, 2H), 1.95-1.48 (m, 16H), 1.25 (s, 46H), 0.86 (d, J=7.2 Hz, 9H).

### Example 3

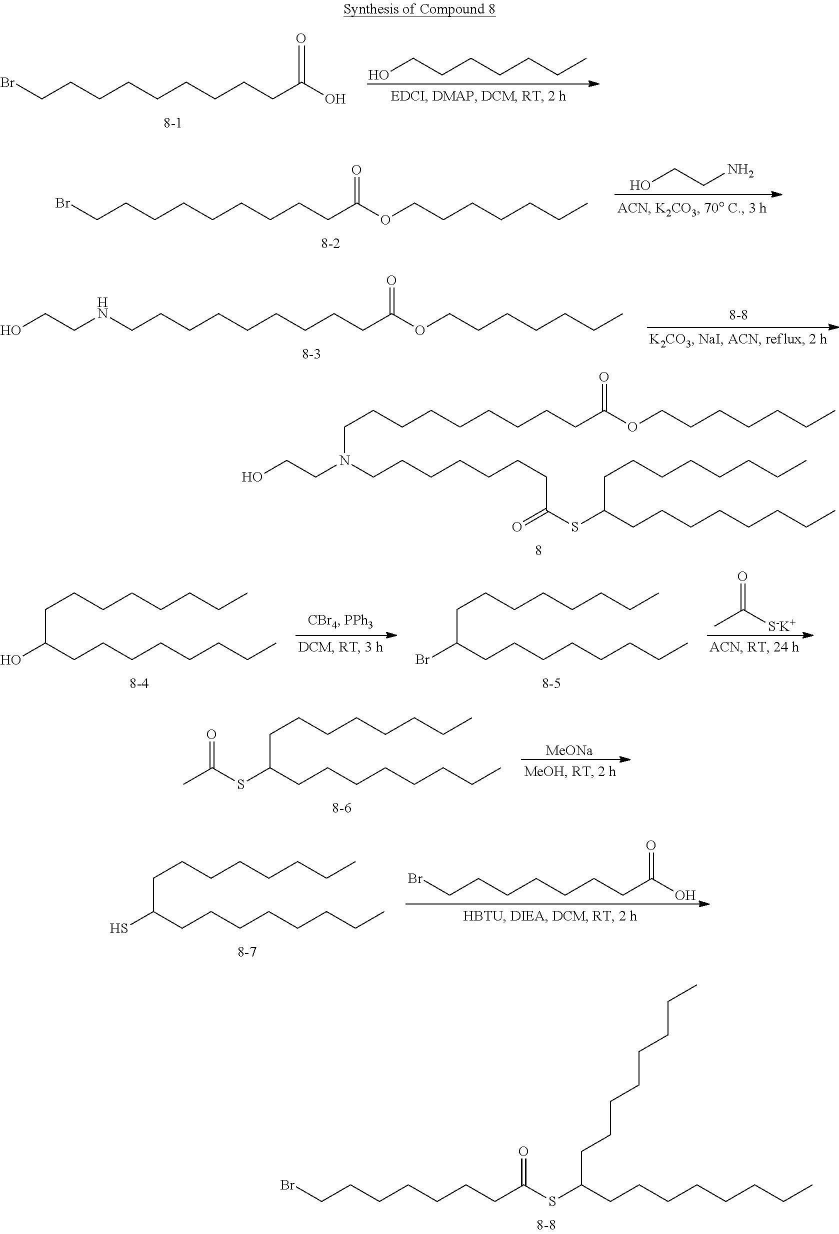

Step 1:

Compound 8-1 (3.0 g) was dissolved in DCM (10 ml) and stirred at room temperature. 1-ethyl-(3-dimethylaminopropyl)carbodiimide hydrochloride (EDCI, 3.43 g), 4-dimethylaminopyridine (DMAP, 1.46 g) and n-heptanol (1.67 g) were weighed sequentially, and added to the reaction system in batches and stirred at room temperature for 2 h. A small amount of the reaction solution was diluted and spotted on the plate in control with a 8-1 standard sample (PE/EA=10/1, phosphomolybdic acid). New spots with reduced polarity were observed. The reaction solution was evaporated under reduced pressure. It was mixed with appropriate amount of silica gel and DCM, and purified (25 g normal phase column, PE/EA, 0-0% 5 min, 0-5% 20 min, 5-5% 5 min, a flow rate of 20 ml/min). The spot plate was monitored, and fractions of the pure product were evaporated to give a colorless oily liquid compound 8-2 (3.5 g, 84% yield).

Step 2:

To a solution of compound 8-2 (3.0 g) and ethanolamine (780 mg) in acetonitrile (100 mL), potassium carbonate (3.6 g) was added. The mixture was stirred at 70° C. for 3 h. TLC showed complete disappearance of compound 8-2 and generation of a point with increased polarity. The reaction solution was filtered. The resulting filtrate was concentrated to obtain a crude product, which was then was mixed with appropriate amount of silica gel and DCM, and purified (25 g normal phase column, PE/EA, 0-0% 5 min, 0-10% 20 min, 10-10% 5 min, a flow rate of 20 ml/min), to give a colorless oily liquid compound 8-3 (1.7 g, 60% yield).

Step 3:

Compound 8-4 (3.0 g) was dissolved in DCM (40 ml) and stirred in ice bath. Triphenylphosphine (2.45 g) and carbon tetrabromide (7.76 g) were weighed sequentially, and added to the reaction system in batches and stirred at room temperature for 2 h. New spots with reduced polarity were observed. The reaction solution was evaporated under reduced pressure. It was mixed with appropriate amount of silica gel and DCM, and purified (40 g normal phase column, PE/EA, 0-0% 5 min, 0-5% 20 min, 5-5% 5 min, a flow rate of 30 ml/min), to give a colorless oily liquid compound 8-5 (2.1 g, 84% yield).

Step 4:

Compound 8-5 (2.0 g) was dissolved in anhydrous acetonitrile (50 ml) and stirred at room temperature. Subsequently, potassium thioacetate (1.43 g) was weighted. It was added to the reaction system in batches and heated and stirred at 85° C. under reflux for 24 h. The reaction solution was concentrated under reduced pressure, and the resulting solid was dissolved in ethyl acetate and then washed three times with saturated brine. The resulting organic phase was evaporated under reduced pressure. The sample was mixed with appropriate amount of silica gel and DCM, and purified (40 g normal phase column, PE/EA, 0-0% 5 min, 0-5% 20 min, 5-5% 5 min, a flow rate of 10 ml/min), to give a pink oily liquid compound 8-6 (1.6 g, 81% yield).

Step 5:

Compound 8-6 (1.5 g) was dissolved in methanol (100 ml) and stirred at room temperature. Subsequently, a solution of sodium methoxide in methanol (1 M) was added to the reaction system and stirred at room temperature under nitrogen atmosphere for 2 h. The reaction solution was neutralized with Amberlite ion exchange resin IR120, filtered and washed, and the organic phase was concentrated. The resulting solid was dissolved in ethyl acetate and then washed three times with saturated brine. The resulting organic phase was evaporated under reduced pressure. It was mixed with appropriate amount of silica gel and DCM, and purified (40 g normal phase column, PE/EA, 0-0% 5 min, 0-5% 20 min, 5-5% 5 min, a flow rate of 10 ml/min), to give a light yellow oily liquid compound 8-7 (1.1 g, 81% yield).

Step 6:

Compound 1-1 (800 mg) was dissolved in DCM (40 ml) and stirred at room temperature. Benzotriazole-N,N,N′,N′-tetramethyluronium hexafluorophosphate (HBTU, 1.6 g), N,N-diisopropylethylamine (DIEA, 930 mg) and compound 8-7 (1.1 g) were weighed sequentially, and added to the reaction system in batches and stirred at room temperature for 2 h. A small amount of the reaction solution was diluted and spotted on the plate in control with a 1-1 standard sample (PE/EA=10/1, phosphomolybdic acid). New spots with reduced polarity were observed. The reaction solution was evaporated under reduced pressure. It was mixed with appropriate amount of silica gel and DCM, and purified (25 g normal phase column, PE/EA, 0-0% 5 min, 0-5% 20 min, 5-5% 5 min, a flow rate of 20 ml/min), to give a colorless oily liquid compound 8-8 (1.4 g, 82% yield).

Step 7:

Compound 8-3 (500 mg) was dissolved in acetonitrile (10 ml) and stirred at room temperature. Subsequently, NaI (228 mg), K₂CO₃ (420 mg) and compound 8-8 (870 mg) were weighed sequentially, and added to the reaction system in batches and heated and stirred at 85° C. under reflux for 2 h. A small amount of the reaction solution was diluted and spotted on the plate (DCM/MeOH=10/1, id aqueous ammonia, phosphomolybdic acid). New spots with less polarity than that of 8-3 were observed. The reaction solution was cooled to room temperature and then evaporated under reduced pressure. It was mixed with appropriate amount of silica gel and DCM, purified (25 g normal phase column, DCM/MeOH, 0.1% aqueous ammonia, 0-0% 10 min, 0-7.5% 20 min, 7.5-7.5% 5 min, a flow rate of 25 ml/min), and concentrated to give a light yellow oily liquid compound 8 (600 mg, 54% yield).

¹H NMR (400 MHz, Chloroform-d) δ 3.60-3.55 (m, 1H), 2.86-2.83 (d, J=7.6 Hz, 2H), 2.81-2.77 (m, 2H), 2.75-2.58 (m, 6H), 2.49-2.44 (m, 2H), 2.31-2.27 (m, 2H), 1.95-1.48 (m, 16H), 1.25 (s, 46H), 0.86 (d, J=7.2 Hz, 9H).

### Example 4

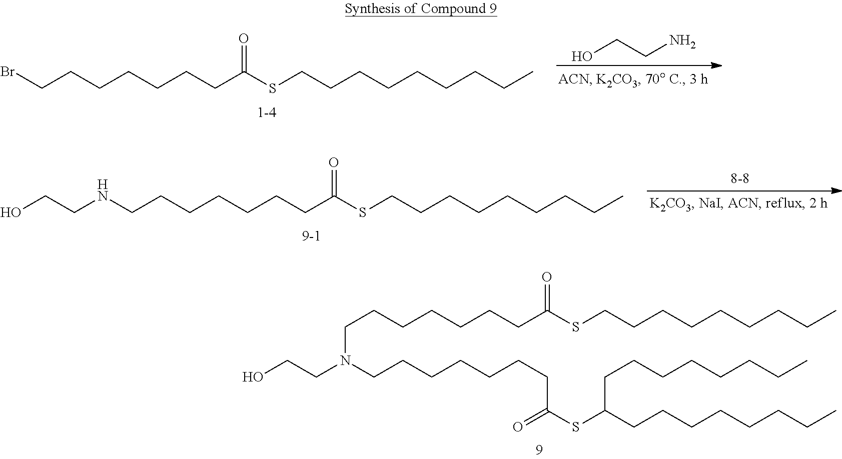

Step 1:

To a solution of compound 1-4 (2.0 g) and ethanolamine (669 mg) in acetonitrile (100 mL), potassium carbonate (2.3 g) was added. The mixture was stirred at 70° C. for 3 h. TLC showed complete disappearance of compound 1-4 and generation of a point with increased polarity. The reaction solution was filtered. The resulting filtrate was concentrated to obtain a crude product, which was then was mixed with appropriate amount of silica gel and DCM, and purified (25 g normal phase column, PE/EA, 0-0% 5 min, 0-10% 20 min, 10-10% 5 min, a flow rate of 20 ml/min), to give a colorless oily liquid compound 8-3 (1.2 g, 63% yield).

Step 2:

Compound 9-1 (500 mg) was dissolved in acetonitrile (10 ml) and stirred at room temperature. Subsequently, NaI (217 mg), K₂CO₃ (400 mg) and compound 8-8 (830 mg) were weighed sequentially, and added to the reaction system in batches and heated and stirred at 85° C. under reflux for 2 h. A small amount of the reaction solution was diluted and spotted on the plate (DCM/MeOH=10/1, id aqueous ammonia, phosphomolybdic acid). New spots with less polarity than that of 9-1 were observed. The reaction solution was cooled to room temperature and then evaporated under reduced pressure. It was mixed with appropriate amount of silica gel and DCM, purified (15 g normal phase column, DCM/MeOH, 0.1% aqueous ammonia, 0-0% 10 min, 0-7.5% 20 min, 7.5-7.5% 5 min, a flow rate of 25 ml/min), and concentrated to give a light yellow oily liquid compound 2 (700 mg, 65% yield).

¹H NMR (400 MHz, Chloroform-d) δ 4.01-3.96 (m, 1H), 2.92-2.88 (m, 2H), 2.81-2.75 (m, 2H), 2.79-2.58 (m, 6H), 2.57-2.40 (m, 2H), 2.31-2.27 (m, 2H), 1.95-1.48 (m, 14H), 1.25 (s, 48H), 0.86 (d, J=7.2 Hz, 9H).

### Example 5

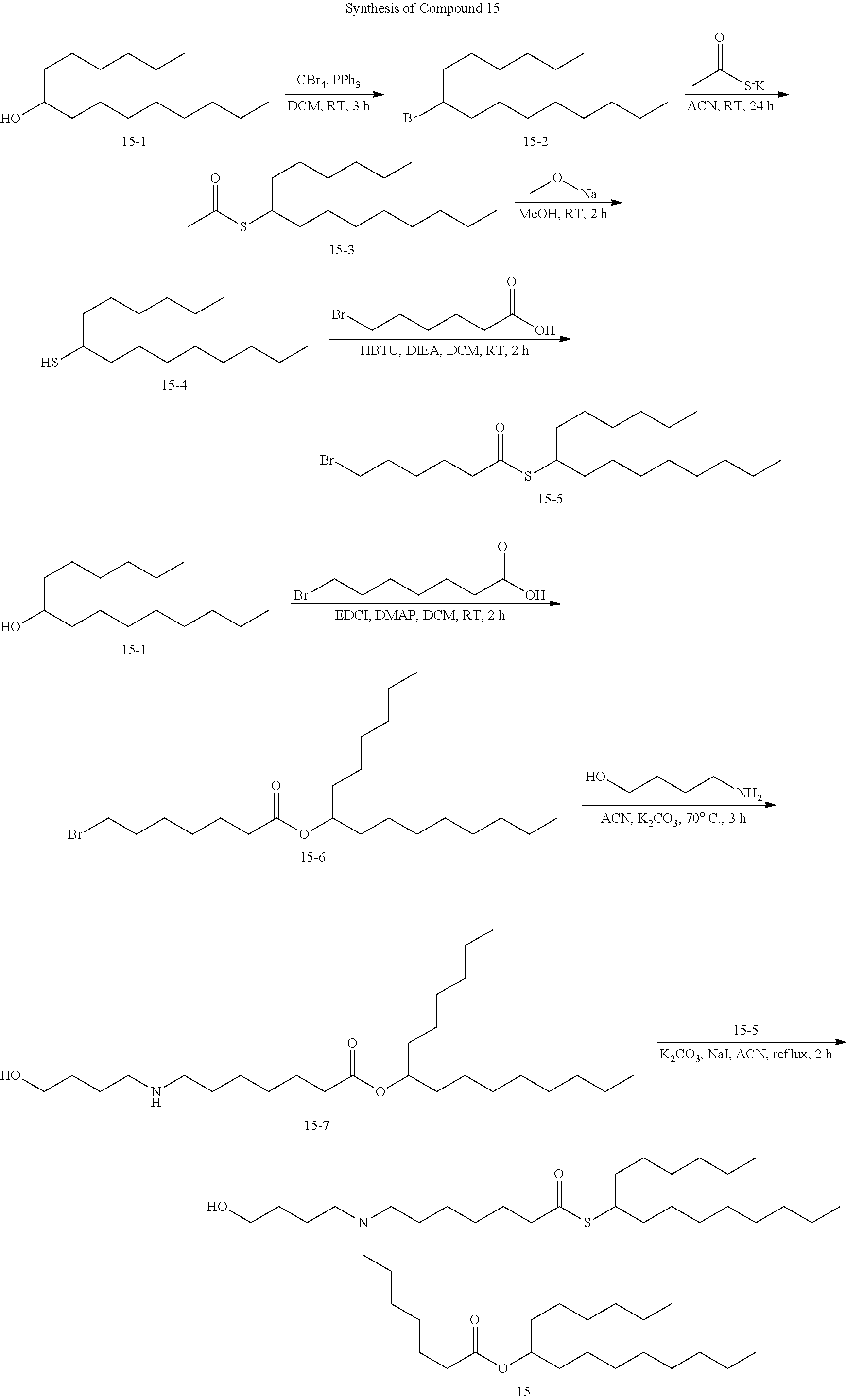

Step 1:

Compound 15-1 (3.0 g) was dissolved in DCM (40 ml) and stirred in ice bath. Triphenylphosphine (2.45 g) and carbon tetrabromide (8.71 g) were weighed sequentially, and added to the reaction system in batches and stirred at room temperature for 2 h. New spots with reduced polarity were observed. The reaction solution was evaporated under reduced pressure. It was mixed with appropriate amount of silica gel and DCM, and purified (40 g normal phase column, PE/EA, 0-0% 5 min, 0-5% 20 min, 5-5% 5 min, a flow rate of 30 ml/min), to give a colorless oily liquid compound 15-2 (3.06 g, 80% yield).

Step 2:

Compound 15-2 (2.0 g) was dissolved in anhydrous acetonitrile (50 ml) and stirred at room temperature. Subsequently, potassium thioacetate (1.57 g) was weighted. It was added to the reaction system in batches and heated and stirred at 85° C. under reflux for 24 h. The reaction solution was concentrated under reduced pressure, and the resulting solid was dissolved in ethyl acetate and then washed three times with saturated brine. The resulting organic phase was evaporated under reduced pressure. It was mixed with appropriate amount of silica gel and DCM, and purified (40 g normal phase column, PE/EA, 0-0% 5 min, 0-5% 20 min, 5-5% 5 min, a flow rate of 10 ml/min), to give a pink oily liquid compound 15-3 (1.49 g, 76% yield).

Step 3:

Compound 15-3 (1.5 g) was dissolved in methanol (100 ml) and stirred at room temperature. Subsequently, 5.2 mL of a solution of sodium methoxide in methanol (1 M) was added to the reaction system in batches and stirred at room temperature under nitrogen atmosphere for 2 h. The reaction solution was neutralized with Amberlite ion exchange resin IR120, filtered and washed, and the organic phase was concentrated. The resulting solid was dissolved in ethyl acetate and then washed three times with saturated brine. The resulting organic phase was evaporated under reduced pressure. It was mixed with appropriate amount of silica gel and DCM, and purified (40 g normal phase column, PE/EA, 0-0% 5 min, 0-5% 20 min, 5-5% 5 min, a flow rate of 10 ml/min), to give a light yellow oily liquid compound 15-4 (1.06 g, 83% yield).

Step 4:

Compound 15-4 (800 mg) was dissolved in DCM (40 ml) and stirred at room temperature. Benzotriazole-N,N,N′,N′-tetramethyluronium hexafluorophosphate (HBTU, 1.79 g), N,N-diisopropylethylamine (DIEA, 1.04 mg) and 6-bromohexanoic acid (1.07 g) were weighed sequentially, and added to the reaction system in batches and stirred at room temperature for 2 h. A small amount of the reaction solution was diluted and spotted on the plate in control with a 15-4 standard sample (PE/EA=10/1, phosphomolybdic acid). New spots with reduced polarity were observed. The reaction solution was evaporated under reduced pressure. It was mixed with appropriate amount of silica gel and DCM, and purified (25 g normal phase column, PE/EA, 0-0% 5 min, 0-5% 20 min, 5-5% 5 min, a flow rate of 20 ml/min), to give a colorless oily liquid compound 15-5 (1.09 g, 79% yield).

Step 5:

7-bromoheptanoic acid (3.0 g) was dissolved in DCM (10 ml) and stirred at room temperature. 1-ethyl-(3-dimethylaminopropyl)carbodiimide hydrochloride (EDCI, 3.15 g), 4-dimethylaminopyridine (DMAP, 1.6 g) and compound 15-1 (2.75 g) were weighed sequentially, and added to the reaction system in batches and stirred at room temperature for 2 h. A small amount of the reaction solution was diluted and spotted on the plate in control with a 15-1 standard sample (PE/EA=10/1, phosphomolybdic acid). New spots with reduced polarity were observed. The reaction solution was evaporated under reduced pressure. It was mixed with appropriate amount of silica gel and DCM, and purified (25 g normal phase column, PE/EA, 0-0% 5 min, 0-5% 20 min, 5-5% 5 min, a flow rate of 20 ml/min). The spot plate was monitored, and fractions of the pure product were evaporated to give a colorless oily liquid compound 15-6 (3.96 g, 72% yield).

Step 6:

To a solution of compound 15-6 (3.0 g) and 4-amino-1-butanol (1.78 mg) in acetonitrile (100 mL), potassium carbonate (2.77 g) was added. The mixture was stirred at 70° C. for 3 h. TLC showed complete disappearance of compound 15-6 and generation of a point with increased polarity. The reaction solution was filtered. The resulting filtrate was concentrated to obtain a crude product, which was then was mixed with appropriate amount of silica gel and DCM, and purified (25 g normal phase column, PE/EA, 0-0% 5 min, 0-10% 20 min, 10-10% 5 min, a flow rate of 20 ml/min), to give a colorless oily liquid compound 15-7 (1.59 g, 52% yield).

Step 7:

Compound 15-7 (500 mg) was dissolved in acetonitrile (10 ml) and stirred at room temperature. Subsequently, NaI (175 mg), K₂CO₃ (485 mg) and compound 15-5 (591 mg) were weighed sequentially, and added to the reaction system in batches and heated and stirred at 85° C. under reflux for 2 h. A small amount of the reaction solution was diluted and spotted on the plate (DCM/MeOH=10/1, Id aqueous ammonia, phosphomolybdic acid). New spots with less polarity than that of 15-7 were observed. The reaction solution was cooled to room temperature and then evaporated under reduced pressure. The sample was mixed with appropriate amount of silica gel and DCM, purified (25 g normal phase column, DCM/MeOH, 0.1% aqueous ammonia, 0-0% 10 min, 0-7.5% 20 min, 7.5-7.5% 5 min, a flow rate of 25 ml/min), and concentrated to give a light yellow oily liquid compound 15 (557 mg, 61% yield).

¹H NMR (400 MHz, Chloroform-d) δ 4.01-3.96 (m, 1H), 2.85 (d, J=7.6 Hz, 4H), 2.81-2.75 (m, 1H), 2.70-2.58 (m, 6H), 2.57-2.40 (m, 2H), 1.95-1.48 (m, 16H), 1.25 (s, 52H), 0.86 (d, J=7.2 Hz, 12H).

### Example 6

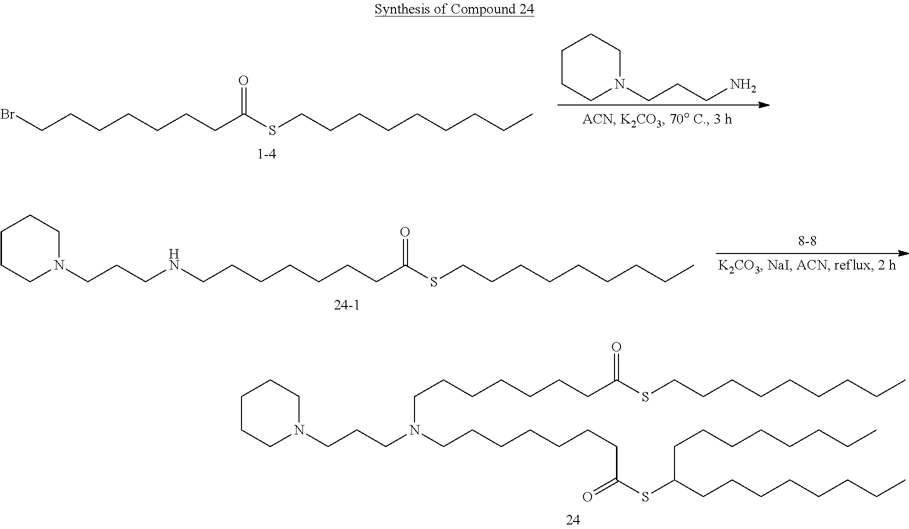

Step 1:

To a solution of compound 1-4 (2.0 g) and 1-(3-aminopropyl)piperidine (2.18 g) in acetonitrile (100 mL), potassium carbonate (2.12 g) was added. The mixture was stirred at 70° C. for 3 h. TLC showed complete disappearance of compound 1-4 and generation of a point with increased polarity. The reaction solution was filtered. The resulting filtrate was concentrated to obtain a crude product, which was then was mixed with appropriate amount of silica gel and DCM, and purified (25 g normal phase column, PE/EA, 0-0% 5 min, 0-10% 20 min, 10-10% 5 min, a flow rate of 20 ml/min), to give a colorless oily liquid compound 24-1 (1.42 g, 61% yield).

Step 2:

Compound 24-1 (500 mg) was dissolved in acetonitrile (10 ml) and stirred at room temperature. Subsequently, NaI (176 mg), K₂CO₃ (486 mg) and compound 8-8 (672 mg) were weighed sequentially, and added to the reaction system in batches and heated and stirred at 85° C. under reflux for 2 h. A small amount of the reaction solution was diluted and spotted on the plate (DCM/MeOH=10/1, id aqueous ammonia, phosphomolybdic acid). New spots with less polarity than that of 24-1 were observed. The reaction solution was cooled to room temperature and then evaporated under reduced pressure. It was mixed with appropriate amount of silica gel and DCM, purified (15 g normal phase column, DCM/MeOH, 0.1% aqueous ammonia, 0-0% 10 min, 0-7.5% 20 min, 7.5-7.5% 5 min, a flow rate of 25 ml/min), and concentrated to give a light yellow oily liquid compound 24 (700 mg, 65% yield).

¹H NMR (400 MHz, Chloroform-d) δ 3.24 (d, J=3.2 Hz, 2H), 3.01 (d, J=4.2 Hz, 2H), 2.79-2.64 (m, 1H), 2.53 (d, J=3.4 Hz, 2H), 2.44 (d, J=3.2 Hz, 2H), 2.33-2.21 (m, 10H), 1.95-1.83 (m, 6H), 1.64-1.51 (m, 16H), 1.42-1.25 (m, 48H), 0.91-0.88 (m, 9H).

### Example 7

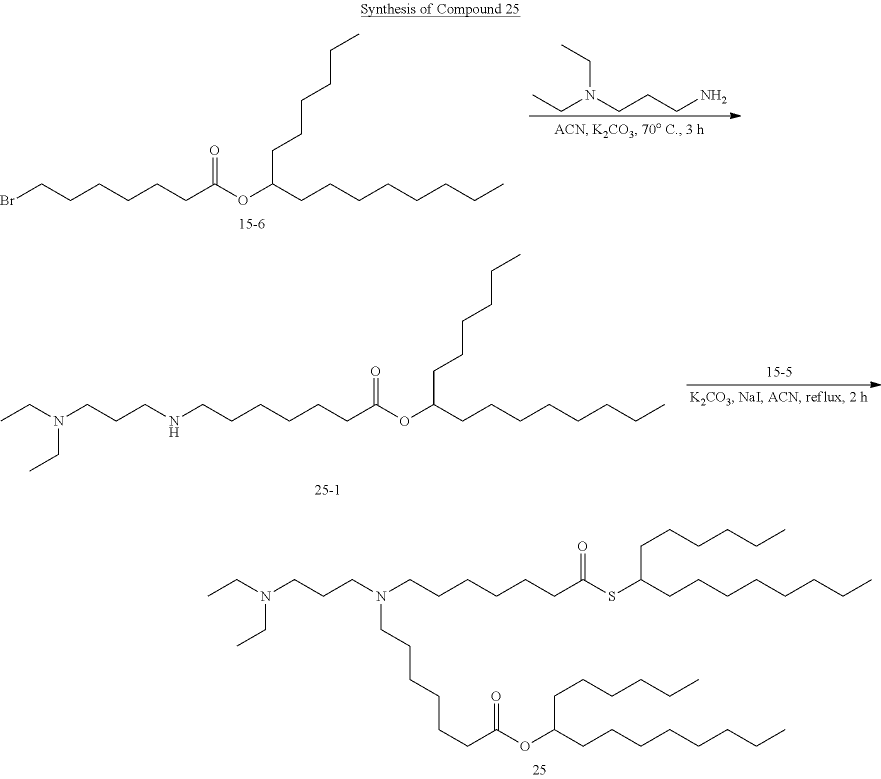

Step 1:

To a solution of compound 15-6 (3.0 g) and 3-diethylaminopropylamine (1.9 g) in acetonitrile (100 mL), potassium carbonate (2.97 g) was added. The mixture was stirred at 70° C. for 3 h. TLC showed complete disappearance of compound 15-6 and generation of a point with increased polarity. The reaction solution was filtered. The resulting filtrate was concentrated to obtain a crude product, which was then was mixed with appropriate amount of silica gel and DCM, and purified (25 g normal phase column, PE/EA, 0-0% 5 min, 0-10% 20 min, 10-10% 5 min, a flow rate of 20 ml/min), to give a colorless oily liquid compound 25-1 (1.8 g, 54% yield).

Step 2:

Compound 25-1 (500 mg) was dissolved in acetonitrile (10 ml) and stirred at room temperature. Subsequently, NaI (160 mg), K₂CO₃ (442 mg) and compound 15-5 (662 mg) were weighed sequentially, and added to the reaction system in batches and heated and stirred at 85° C. under reflux for 2 h. A small amount of the reaction solution was diluted and spotted on the plate (DCM/MeOH=10/1, id aqueous ammonia, phosphomolybdic acid). New spots with less polarity than that of 15-5 were observed. The reaction solution was cooled to room temperature and then evaporated under reduced pressure. It was mixed with appropriate amount of silica gel and DCM, purified (25 g normal phase column, DCM/MeOH, 0.1% aqueous ammonia, 0-0% 10 min, 0-7.5% 20 min, 7.5-7.5% 5 min, a flow rate of 25 ml/min), and concentrated to give a light yellow oily liquid compound 25 (150 mg, 17% yield).

¹H NMR (400 MHz, Chloroform-d) δ 4.81-4.82 (m, 1H), 3.89 (d, J=7.2 Hz, 4H), 3.23-3.12 (m, 1H), 3.02 (d, J=3.4 Hz, 2H), 2.48 (d, J=7.2 Hz, 4H), 2.36 (d, J=3.4 Hz, 2H), 2.31 (d, J=3.2 Hz, 2H), 2.07-1.86 (m, 4H), 1.65-1.56 (m, 4H), 1.53-1.43 (m, 6H), 1.41-1.19 (m, 54H), 1.04 (d, J=7.8 Hz, 6H), 0.85 (d, J=12.4 Hz, 12H).

### Example 8

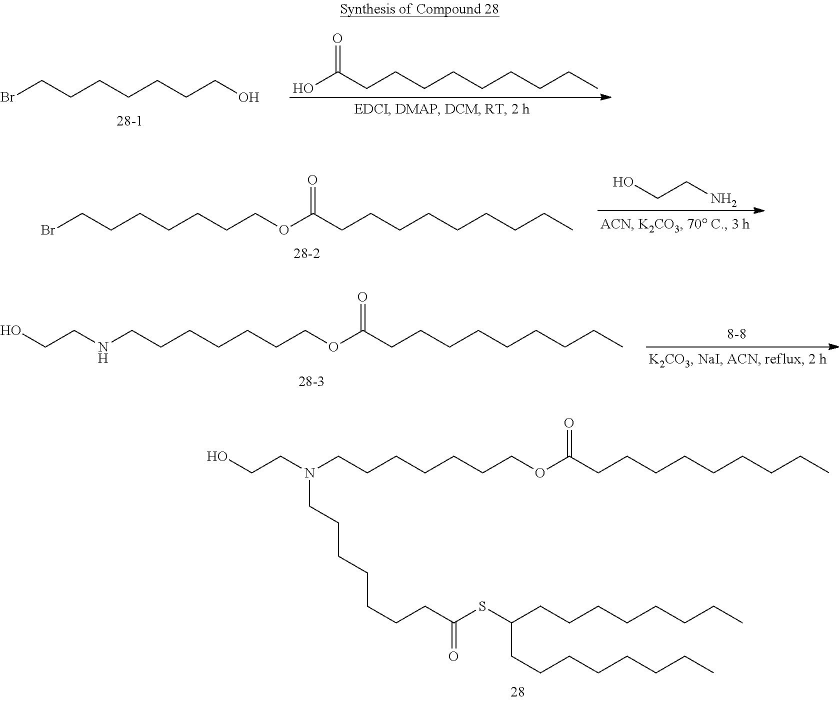

Step 1:

Compound 28-1 (2.5 g) was dissolved in DCM (10 ml) and stirred at room temperature. 1-ethyl-(3-dimethylaminopropyl)carbodiimide hydrochloride (EDCI, 2.9 g), 4-dimethylaminopyridine (DMAP, 1.42 g) and n-heptanol (2.5 g) were weighed sequentially, and added to the reaction system in batches and stirred at room temperature for 2 h. The reaction solution was evaporated under reduced pressure. It was mixed with appropriate amount of silica gel and DCM, and purified (25 g normal phase column, PE/EA, 0-0% 5 min, 0-5% 20 min, 5-5% 5 min, a flow rate of 20 ml/min). The spot plate was monitored, and fractions of the pure product were evaporated to give a colorless oily liquid compound 28-2 (3.1 g, 76% yield).

Step 2:

To a solution of compound 28-2 (3.0 g) and ethanolamine (700 mg) in acetonitrile (100 mL), potassium carbonate (2.4 g) was added. The mixture was stirred at 70° C. for 3 h. TLC showed complete disappearance of compound 28-2 and generation of a point with increased polarity. The reaction solution was filtered. The resulting filtrate was concentrated to obtain a crude product, which was then was mixed with appropriate amount of silica gel and DCM, and purified (25 g normal phase column, PE/EA, 0-0% 5 min, 0-10% 20 min, 10-10% 5 min, a flow rate of 20 ml/min), to give a colorless oily liquid compound 28-3 (1.42 g, 75% yield).

Step 3:

Compound 28-3 (500 mg) was dissolved in acetonitrile (10 ml) and stirred at room temperature. Subsequently, NaI (228 mg), K₂CO₃ (630 mg) and compound 8-8 (1.1 g) were weighed sequentially, and added to the reaction system in batches and heated and stirred at 85° C. under reflux for 2 h. A small amount of the reaction solution was diluted and spotted on the plate (DCM/MeOH=10/1, Id aqueous ammonia, phosphomolybdic acid). New spots with less polarity than that of 28-3 were observed. The reaction solution was cooled to room temperature and then evaporated under reduced pressure. It was mixed with appropriate amount of silica gel and DCM, purified (25 g normal phase column, DCM/MeOH, 0.1% aqueous ammonia, 0-0% 10 min, 0-7.5% 20 min, 7.5-7.5% 5 min, a flow rate of 25 ml/min), and concentrated to give a light yellow oily liquid compound 28 (450 mg, 41% yield).

¹H NMR (400 MHz, Chloroform-d) δ 4.13 (d, J=3.2 Hz, 2H), 3.45 (d, J=3.4 Hz, 2H), 3.01 (d, J=7.2 Hz, 4H), 2.87-2.76 (m, 1H), 2.53 (d, J=3.2 Hz, 2H), 2.40 (d, J=3.2 Hz, 2H), 2.32 (d, J=3.2 Hz, 2H), 1.98-1.88 (m, 4H), 1.77-1.63 (m, 6H), 1.54-1.44 (m, 6H), 1.42-1.26 (m, 46H), 0.88 (d, J=9.8 Hz, 9H).

### Example 9

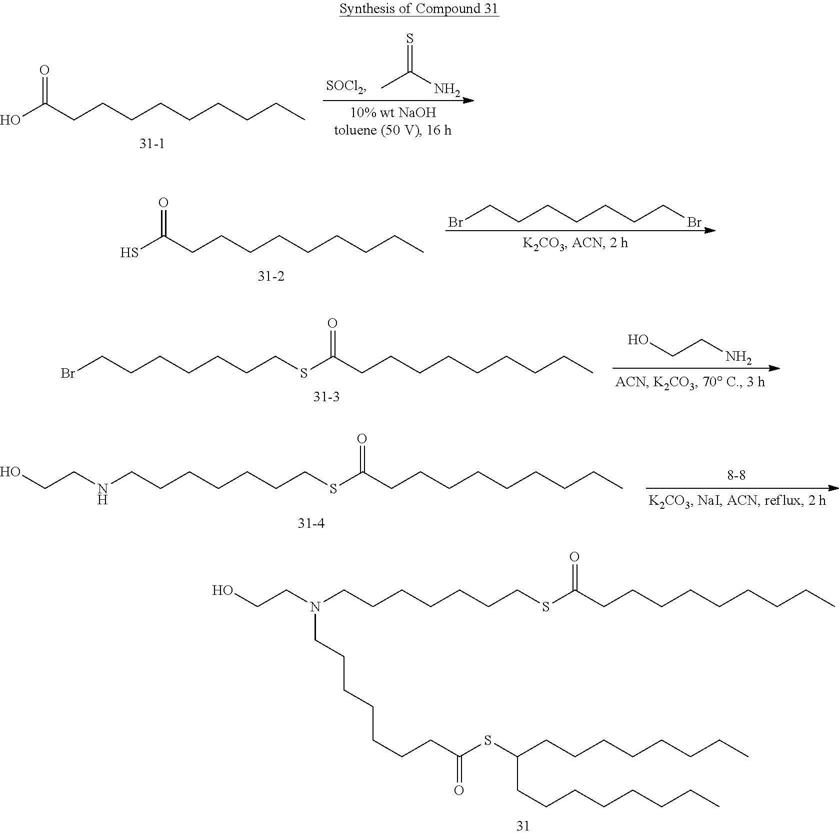

Step 1:

Raw material 31-1 (5 g) was dissolved in DCM (50 ml), a drop of DMF was added thereto, and the gas was replaced with N₂. Subsequently SOC₂ (4.14 g) was weighed and added dropwise to the above reaction solution and stirred at room temperature for 3 h. A small amount of the reaction solution was diluted and spotted on the plate in control with a 31-1 standard sample (PE/EA=10/1, phosphomolybdic acid). New spots with less polarity than that of 31-1 were observed, with less 31-1 remaining. The reaction solution was spun-dried under reduced pressure, and dissolved in a small amount of DCM. It was then added dropwise to a solution of thioacetamide (4.36 g) in toluene (50 mL), and heated and stirred at 40° C. for 3 h. A solution of 10% NaOH (30 ml) was added dropwise to the reaction system, and heated and stirred at 40° C. overnight. A small amount of the reaction solution was diluted and spotted on the plate (PE/EA=20/1, phosphomolybdic acid), and several trailing main spots was observed with UV absorption. The pH of the reaction solution was adjusted to 3-5 with 6 M/L HCl. The reaction solution was extracted with ethyl acetate, and the organic phase was dried and evaporated under reduced pressure. The sample was mixed with appropriate amount of DCM and silica gel and passed through a column (PE, 600 ml), followed by spotting and UV lamp detection, to give a light yellow oily liquid 31-2 (3.02 g, 55% yield).

Step 2:

To a solution of 1,7-dibromoheptane (3.29 g) in tetrahydrofuran (40 mL), compound 31-2 (2.0 g) and potassium carbonate (2.94 g) were added in batches. The mixture was stirred at 45° C. for 3 h. TLC showed complete disappearance of compound 31-2 and generation of a point with increased polarity. The reaction solution was filtered. The resulting filtrate was concentrated to obtain a crude product, which was then was mixed with appropriate amount of silica gel and DCM, and purified (25 g normal phase column, PE/EA, 0-0% 5 min, 0-10% 20 min, 10-10% 5 min, a flow rate of 20 ml/min), to give a colorless oily liquid compound 31-3 (2.33 g, 60% yield).

Step 3:

To a solution of compound 31-2 (2.0 g) and ethanolamine (1.0 g) in acetonitrile (40 mL), potassium carbonate (2.27 g) was added. The mixture was stirred at 70° C. for 3 h. TLC showed complete disappearance of compound 31-3 and generation of a point with increased polarity. The reaction solution was filtered. The resulting filtrate was concentrated to obtain a crude product, which was then was mixed with appropriate amount of silica gel and DCM, and purified (25 g normal phase column, PE/EA, 0-0% 5 min, 0-10% 20 min, 10-10% 5 min, a flow rate of 20 ml/min), to give a colorless oily liquid compound 31-4 (0.92 g, 49% yield).

Step 4:

Compound 31-4 (500 mg) was dissolved in acetonitrile (10 ml) and stirred at room temperature. Subsequently, NaI (217 mg), K₂CO₃ (600 mg) and compound 8-8 (829 mg) were weighed sequentially, and added to the reaction system in batches and heated and stirred at 85° C. under reflux for 2 h. A small amount of the reaction solution was diluted and spotted on the plate (DCM/MeOH=10/1, id aqueous ammonia, phosphomolybdic acid). New spots with less polarity than that of 31-4 were observed. The reaction solution was cooled to room temperature and then evaporated under reduced pressure. The sample was mixed with appropriate amount of silica gel and DCM, purified (25 g normal phase column, DCM/MeOH, 0.1% aqueous ammonia, 0-0% 10 min, 0-7.5% 20 min, 7.5-7.5% 5 min, a flow rate of 25 ml/min), and concentrated to give a light yellow oily liquid compound 31 (647 mg, 60% yield).

¹H NMR (400 MHz, Chloroform-d) δ 3.63 (d, J=3.2 Hz, 2H), 3.23 (d, J=3.4 Hz, 2H), 3.06 (d, J=7.2 Hz, 4H), 2.91-2.85 (m, 1H), 2.57 (d, J=3.2 Hz, 2H), 2.38 (d, J=7.6 Hz, 4H), 2.07-1.86 (m, 6H), 1.74-1.63 (m, 4H), 1.59-1.46 (m, 4H), 1.42-1.26 (m, 48H), 0.87 (d, J=9.8 Hz, 9H).

### Example 10

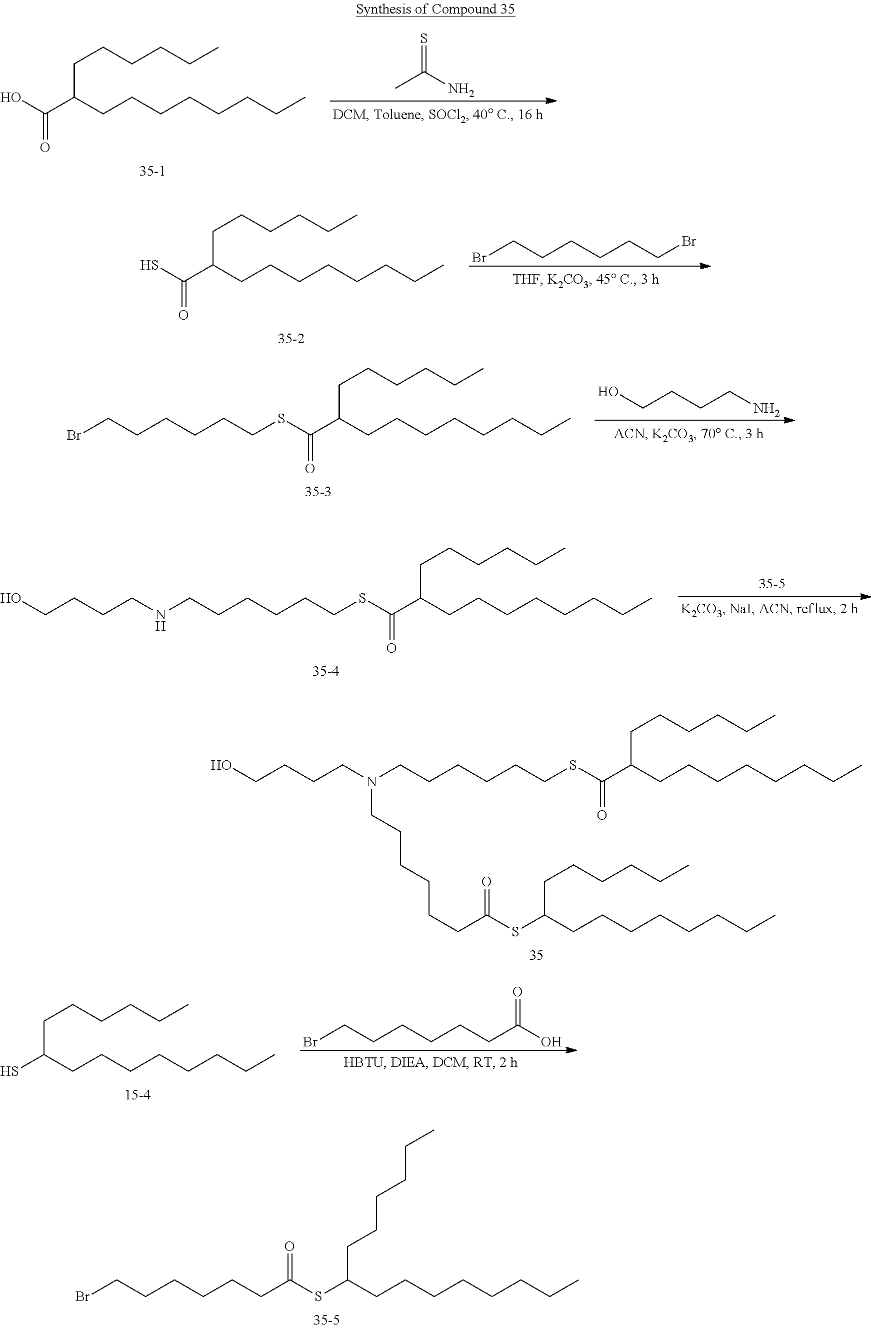

Step 1:

Raw material 35-1 (5 g) was dissolved in DCM (50 ml), a drop of DMF was added thereto, and the gas was replaced with N₂. Subsequently SOC₂ (2.8 g) was weighed and added dropwise to the above reaction solution and stirred at room temperature for 3 h. A small amount of the reaction solution was diluted and spotted on the plate in control with a 35-1 standard sample (PE/EA=10/1, phosphomolybdic acid). New spots with less polarity than that of 35-1 were observed, with less 35-1 remaining. The reaction solution was spun-dried under reduced pressure, and dissolved in a small amount of DCM. It was then added dropwise to a solution of thioacetamide (2.2 g) in toluene (50 mL), and heated and stirred at 40° C. for 3 h. A solution of 10% NaOH (30 ml) was added dropwise to the reaction system, and heated and stirred at 40° C. overnight. A small amount of the reaction solution was diluted and spotted on the plate (PE/EA=20/1, phosphomolybdic acid), and several trailing main spots was observed with UV absorption. The pH of the reaction solution was adjusted to 3-5 with 6 M/L HCl. The reaction solution was extracted with ethyl acetate, and the organic phase was dried and evaporated under reduced pressure. The sample was mixed with appropriate amount of DCM and silica gel and passed through a column (PE, 600 ml), followed by spotting and UV lamp detection, to give a light yellow oily liquid 35-2 (3.1 g, 58% yield).

Step 2:

To a solution of 1,6-dibromohexane (2.15 g) in tetrahydrofuran (40 mL), compound 35-2 (2.0 g) and potassium carbonate (2.03 g) were added in batches. The mixture was stirred at 45° C. for 3 h. TLC showed complete disappearance of compound 35-2 and generation of a point with increased polarity. The reaction solution was filtered. The resulting filtrate was concentrated to obtain a crude product, which was then was mixed with appropriate amount of silica gel and DCM, and purified (25 g normal phase column, PE/EA, 0-0% 5 min, 0-10% 20 min, 10-10% 5 min, a flow rate of 20 ml/min), to give a colorless oily liquid compound 35-3 (2.1 g, 65% yield).

Step 3:

To a solution of compound 35-3 (2.0 g) and 4-amino-1-butanol (1.15 g) in acetonitrile (40 mL), potassium carbonate (1.78 g) was added. The mixture was stirred at 70° C. for 3 h. TLC showed complete disappearance of compound 35-3 and generation of a point with increased polarity. The reaction solution was filtered. The resulting filtrate was concentrated to obtain a crude product, which was then was mixed with appropriate amount of silica gel and DCM, and purified (25 g normal phase column, PE/EA, 0-0% 5 min, 0-10% 20 min, 10-10% 5 min, a flow rate of 20 ml/min), to give a colorless oily liquid compound 35-4 (1.02 g, 50% yield).

Step 4:

Compound 15-4 (800 mg) was dissolved in DCM (40 ml) and stirred at room temperature. Benzotriazole-N,N,N′,N′-tetramethyluronium hexafluorophosphate (HBTU, 1.81 g), N,N-diisopropylethylamine (DIEA, 0.99 mg) and 7-bromoheptanoic acid (1.03 g) were weighed sequentially, and added to the reaction system in batches and stirred at room temperature for 2 h. A small amount of the reaction solution was diluted and spotted on the plate in control with a 35-4 standard sample (PE/EA=10/1, phosphomolybdic acid). New spots with reduced polarity were observed. The reaction solution was evaporated under reduced pressure. The sample was mixed with appropriate amount of silica gel and DCM, and purified (25 g normal phase column, PE/EA, 0-0% 5 min, 0-5% 20 min, 5-5% 5 min, a flow rate of 20 ml/min), to give a colorless oily liquid compound 35-5 (1.03 g, 61% yield).

Step 5:

Compound 35-4 (500 mg) was dissolved in acetonitrile (10 ml) and stirred at room temperature. Subsequently, NaI (169 mg), K₂CO₃ (467 mg) and compound 35-5 (589 mg) were weighed sequentially, and added to the reaction system in batches and heated and stirred at 85° C. under reflux for 2 h. A small amount of the reaction solution was diluted and spotted on the plate (DCM/MeOH=10/1, id aqueous ammonia, phosphomolybdic acid). New spots with less polarity than that of 35-4 were observed. The reaction solution was cooled to room temperature and then evaporated under reduced pressure. It was mixed with appropriate amount of silica gel and DCM, purified (25 g normal phase column, DCM/MeOH, 0.1% aqueous ammonia, 0-0% 10 min, 0-7.5% 20 min, 7.5-7.5% 5 min, a flow rate of 25 ml/min), and concentrated to give a light yellow oily liquid compound 35 (620 mg, 68% yield).

¹H NMR (400 MHz, Chloroform-d) δ 3.51 (d, J=3.2 Hz, 2H), 3.04 (d, J=9.8 Hz, 6H), 2.86 (d, J=3.4 Hz, 2H), 2.71-2.63 (m, 1H), 2.46 (d, J=3.2 Hz, 2H), 2.39-2.28 (m, 1H), 2.01-1.86 (m, 6H), 1.63-1.52 (m, 6H), 1.42-1.26 (m, 56H), 0.87 (d, J=12.8 Hz, 12H).

### Example 11

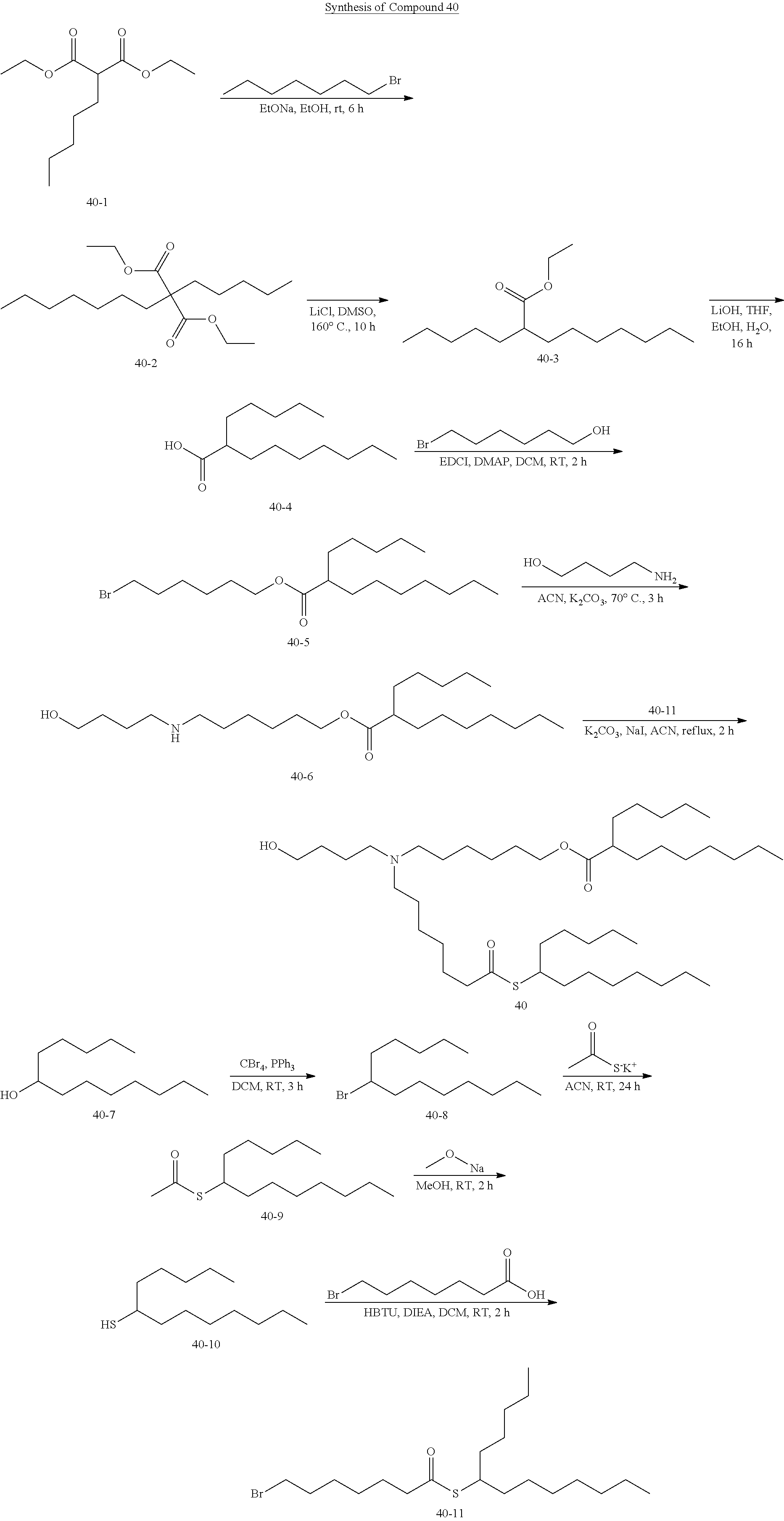

Step 1:

Compound 40-1 (3.0 g) was dissolved in DCM (50 ml) and stirred in ice bath. Sodium ethoxide (EtONa, 1.33 g) and 1-bromoheptane (2.8 g) were weighed sequentially, and added to the reaction system in batches and stirred at room temperature for 6 h. The reaction solution was quenched with saturated ammonium chloride solution and extracted with ethyl acetate, and the resulting organic phase was dried over anhydrous sodium sulfate. It was mixed with appropriate amount of silica gel and DCM, and purified (25 g normal phase column, PE/EA, 0-0% 5 min, 0-5% 20 min, 5-5% 5 min, a flow rate of 20 ml/min). The spot plate was monitored, and fractions of the pure product were evaporated to give a colorless oily liquid compound 40-2 (2.0 g, 47% yield).

Step 2:

To a solution of compound 40-2 (2.0 g) in DMSO (30 mL), lithium chloride (2.1 g) was added. The mixture was stirred at 160° C. for 10 h. The reaction solution was cooled down to room temperature, quenched with water and extracted with ethyl acetate. The resulting organic phase was washed with saturated saline and dried over anhydrous sodium sulfate. Subsequently, it was mixed with appropriate amount of silica gel and purified (25 g normal phase column, PE/EA, 0-0% 5 min, 0-5% 20 min, 5-5% 5 min, a flow rate of 20 ml/min). The spot plate was monitored, and fractions of the pure product were evaporated to give a colorless oily liquid compound 40-3 (800 mg, 51% yield).

Step 3:

Compound 40-3 (800 mg) was dissolved in ethanol (10 mL) and tetrahydrofuran (10 ml). Subsequently, a solution of lithium hydroxide (748 mg) in water (10 mL) was added and stirred at room temperature for 16 h. After adding water and ethyl acetate, the aqueous phase was adjusted to pH=2 with dilute hydrochloric acid, and extracted three times with ethyl acetate. The resulting organic phase was dried over anhydrous sodium sulfate and concentrated to give a colorless oily liquid compound 40-4 (660 mg, 92% yield).

Step 4:

Compound 40-4 (1.0 g) was dissolved in DCM (20 ml) and stirred at room temperature. 1-ethyl-(3-dimethylaminopropyl)carbodiimide hydrochloride (EDCI, 1.1 g), 4-dimethylaminopyridine (DMAP, 535 mg) and 6-bromo-1-hexanol (873 mg) were weighed sequentially, and added to the reaction system in batches and stirred at room temperature for 2 h. The reaction solution was evaporated under reduced pressure. It was mixed with appropriate amount of silica gel and DCM, and purified (25 g normal phase column, PE/EA, 0-0% 5 min, 0-5% 20 min, 5-5% 5 min, a flow rate of 20 ml/min). The spot plate was monitored, and fractions of the pure product were evaporated to give a colorless oily liquid compound 40-5 (1.3 g, 76% yield).

Step 5:

To a solution of compound 40-5 (1.2 g) and 4-amino-1-butanol (820 mg) in acetonitrile (100 mL), potassium carbonate (1.3 g) was added. The mixture was stirred at 70° C. for 3 h. TLC showed complete disappearance of compound 40-5 and generation of a point with increased polarity. The reaction solution was filtered. The resulting filtrate was concentrated to obtain a crude product, which was then was mixed with appropriate amount of silica gel and DCM, and purified (25 g normal phase column, PE/EA, 0-0% 5 min, 0-10% 20 min, 10-10% 5 min, a flow rate of 20 ml/min), to give a colorless oily liquid compound 40-6 (800 mg, 65% yield).

Step 6:

Compound 40-7 (3.0 g) was dissolved in DCM (40 ml) and stirred in ice bath. Triphenylphosphine (2.76 g) and carbon tetrabromide (8.71 g) were weighed sequentially, and added to the reaction system in batches and stirred at room temperature for 2 h. New spots with reduced polarity were observed. The reaction solution was evaporated under reduced pressure. It was mixed with appropriate amount of silica gel and DCM, and purified (40 g normal phase column, PE/EA, 0-0% 5 min, 0-5% 20 min, 5-5% 5 min, a flow rate of 30 ml/min), to give a colorless oily liquid compound 40-8 (2.6 g, 66% yield).

Step 7:

Compound 40-8 (2.0 g) was dissolved in anhydrous acetonitrile (50 ml) and stirred at room temperature. Subsequently, potassium thioacetate (1.57 g) was weighted. It was added to the reaction system and heated and stirred at 85° C. under reflux for 24 h. The reaction solution was concentrated under reduced pressure, and the resulting solid was dissolved in ethyl acetate and then washed three times with saturated brine. The resulting organic phase was evaporated under reduced pressure. It was mixed with appropriate amount of silica gel, and purified (40 g normal phase column, PE/EA, 0-0% 5 min, 0-5% 20 min, 5-5% 5 min, a flow rate of 10 ml/min), to give a pink oily liquid compound 40-9 (1.5 g, 76% yield).

Step 8:

Compound 40-9 (1.5 g) was dissolved in methanol (100 ml) and stirred at room temperature. Subsequently, a solution of sodium methoxide (6.0 mL) in methanol (1 M) was added to the reaction system and stirred at room temperature under nitrogen atmosphere for 2 h. The reaction solution was neutralized with Amberlite ion exchange resin IR120, filtered and washed, and the organic phase was concentrated. The resulting solid was dissolved in ethyl acetate and then washed three times with saturated brine. The resulting organic phase was evaporated under reduced pressure. It was mixed with appropriate amount of silica gel, and purified (40 g normal phase column, PE/EA, 0-0% 5 min, 0-5% 20 min, 5-5% 5 min, a flow rate of 10 ml/min), to give a light yellow oily liquid compound 40-10 (950 mg, 76% yield).

Step 9:

Compound 40-10 (800 mg) was dissolved in DCM (40 ml) and stirred at room temperature. Benzotriazole-N,N,N′,N′-tetramethyluronium hexafluorophosphate (HBTU, 1.79 g), N,N-diisopropylethylamine (DIEA, 1.04 g) and 7-bromohexanoic acid (1.1 g) were weighed sequentially, and added to the reaction system in batches and stirred at room temperature for 2 h. A small amount of the reaction solution was diluted and spotted on the plate in control with a 40-10 standard sample (PE/EA=10/1, phosphomolybdic acid). New spots with reduced polarity were observed. The reaction solution was evaporated under reduced pressure. The sample was mixed with appropriate amount of silica gel and DCM, and purified (25 g normal phase column, PE/EA, 0-0% 5 min, 0-5% 20 min, 5-5% 5 min, a flow rate of 20 ml/min), to give a colorless oily liquid compound 40-11 (1.20 g, 80% yield).

Step 10:

Compound 40-6 (500 mg) was dissolved in acetonitrile (10 ml) and stirred at room temperature. Subsequently, NaI (188 mg), K₂CO₃ (519 mg) and compound 8-8 (817 mg) were weighed sequentially, and added to the reaction system in batches and heated and stirred at 85° C. under reflux for 2 h. A small amount of the reaction solution was diluted and spotted on the plate (DCM/MeOH=10/1, Id aqueous ammonia, phosphomolybdic acid). New spots with less polarity than that of 40-6 were observed. The reaction solution was cooled to room temperature and then evaporated under reduced pressure. It was mixed with appropriate amount of silica gel and DCM, purified (25 g normal phase column, DCM/MeOH, 0.1% aqueous ammonia, 0-0% 10 min, 0-7.5% 20 min, 7.5-7.5% 5 min, a flow rate of 25 ml/min), and concentrated to give a light yellow oily liquid compound 40 (600 mg, 66% yield).

¹H NMR (400 MHz, Chloroform-d) δ 4.01-3.96 (m, 1H), 2.85 (d, J=7.6 Hz, 4H), 2.81-2.75 (m, 1H), 2.70-2.58 (m, 6H), 2.57-2.40 (m, 2H), 1.95-1.48 (m, 14H), 1.25 (s, 46H), 0.86 (d, J=7.2 Hz, 12H).

### Example 12

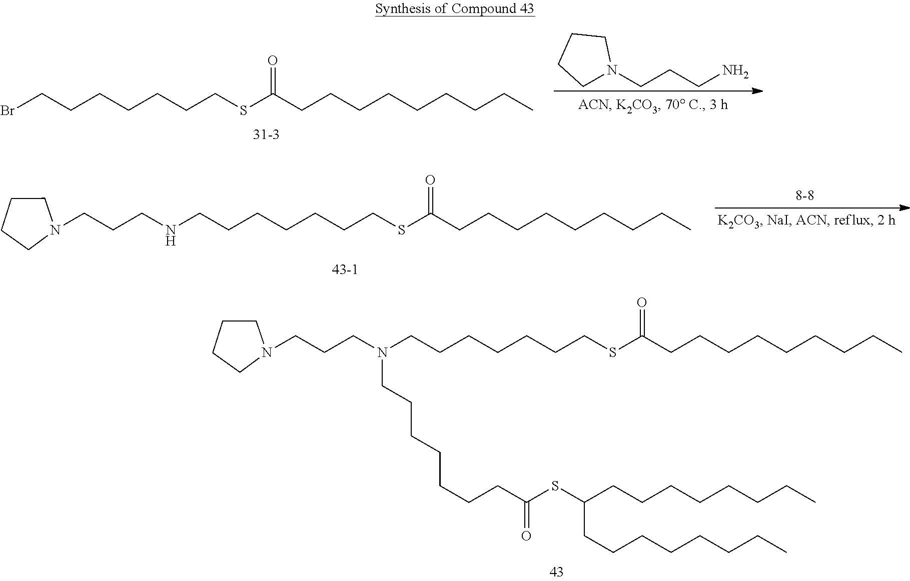 [SMILES: CCCCCCCCCC(=O)SCCCCCCCBr ; NCCCN1CCCC1 ; CCCCCCCCCC(=O)SCCCCCCCNCCCN1CCCC1 ; CCCCCCCCCC(=O)SCCCCCCCN(CCCCCCCC(=O)SC(CCCCCCCC)CCCCCCCC)CCCN1CCCC1]

Step 1:

To a solution of compound 31-3 (3.0 g) and 1-(3-aminopropyl)pyrrolidine (2.11 g) in acetonitrile (100 mL), potassium carbonate (2.27 g) was added. The mixture was stirred at 70° C. for 3 h. TLC showed complete disappearance of compound 31-3 and generation of a point with increased polarity. The reaction solution was filtered. The resulting filtrate was concentrated to obtain a crude product, which was then was mixed with appropriate amount of silica gel and DCM, and purified (25 g normal phase column, PE/EA, 0-0% 5 min, 0-10% 20 min, 10-10% 5 min, a flow rate of 20 ml/min), to give a colorless oily liquid compound 43-1 (1.10 g, 49% yield).

Step 2:

Compound 43-1 (500 mg) was dissolved in acetonitrile (10 ml) and stirred at room temperature. Subsequently, NaI (181 mg), K₂CO₃ (502 mg) and compound 8-8 (695 mg) were weighed sequentially, and added to the reaction system in batches and heated and stirred at 85° C. under reflux for 2 h. A small amount of the reaction solution was diluted and spotted on the plate (DCM/MeOH=10/1, id aqueous ammonia, phosphomolybdic acid). New spots with less polarity than that of 43-1 were observed. The reaction solution was cooled to room temperature and then evaporated under reduced pressure. It was mixed with appropriate amount of silica gel and DCM, purified (15 g normal phase column, DCM/MeOH, 0.1% aqueous ammonia, 0-0% 10 min, 0-7.5% 20 min, 7.5-7.5% 5 min, a flow rate of 25 ml/min), and concentrated to give a light yellow oily liquid compound 43 (300 mg, 31% yield).

¹H NMR (400 MHz, Chloroform-d) δ 3.24 (d, J=3.2 Hz, 2H), 3.01 (d, J=4.2 Hz, 2H), 2.79-2.64 (m, 1H), 2.53 (d, J=3.4 Hz, 2H), 2.44 (d, J=3.2 Hz, 2H), 2.33-2.21 (m, 10H), 1.95-1.83 (m, 6H), 1.64-1.51 (m, 16H), 1.42-1.25 (m, 46H), 0.91-0.88 (m, 9H).

### Example 13—Preparation of Nano-Lipid Particles and Characterization of Properties Thereof

Luciferase mRNA (Luc mRNA) was diluted in 10-100 mM citrate buffer at pH 4.0; lipid components (cationic lipids indicated in the present disclosure: DSPC:cholesterol:PEG lipids) were dissolved in ethanol at a total concentration of 10 mg/mL, wherein ALC-0159 was used for the group of 1.6% PEG lipid, and DMG-PEG2000 was used for the group of 1.5% PEG lipid.

3 mL of a mRNA buffer and 1 mL of a lipid solution were loaded into two 5 mL syringes respectively, and the syringes were installed on a microfluidic syringe pump. Chips were connected to the syringes while the syringe pump flow rate was set. Clicking the start button of the syringe pump caused injection into the chip at a flow rate ratio of 3:1. Under observation of the color of the product at the exit of the chip, after discarding the first 5 milky white droplets (about 100 μL), the back-end sample was collected into an EP tube. The collected product was placed in a dialysis bag and dialyzed for 6 h (MWCO: 100 KDa) in 10 mM PBS (pH 7.4) intervals. Subsequently, after being concentrated by ultrafiltration to the desired concentration, the lipid nanoparticles were filtered through a 0.22 μm sterile filter and then stored at 4° C.

Tests were performed to calculate the encapsulation rate of the products according to the instructions of Ribogreen kit. Particle size and polydispersity index (PDI) detection as well as zeta potential analysis was performed on a Malvern Zetasizer nano instrument using standard assays.

The results of particle size, PDI and encapsulation rate of LNP loaded with mRNA prepared in this example are shown in Table 1. The results indicate that the nanoparticles formed by lipids and mRNA under this formulation had a high encapsulation rate and a uniform particle size of about 100 nm, which conformed to the basic characteristics of carriers for delivering nucleic acids.

TABLE 1

| | | Lipid molar ratio | | Particle | | Zeta |
|---|---|---|---|---|---|---|
| | Cationic | Cationic:DSPC:cholesterol:PEG | Encapsulation | size | | potential |
| No. | lipid | lipid | rate (%) | (nm) | PDI | (mV) |
| 1 | Compound 1 | 50:10:38.5:1.5 | 97.5 | 103.2 | 0.24 | −0.05 |
| 2 | Compound 8 | 50:10:38.5:1.5 | 91.4 | 88.5 | 0.19 | 0.25 |
| 3 | Compound 9 | 50:10:38.5:1.5 | 95.7 | 101.7 | 0.12 | −0.63 |
| 4 | Compound 15 | 46.3:9.4:42.7:1.6 | 99.1 | 99.5 | 0.24 | −2.76 |
| 5 | Compound 25 | 45:15:38.5:1.5 | 93.8 | 108.6 | 0.21 | 3.63 |
| 6 | Compound 31 | 50:10:38.5:1.5 | 94.1 | 98.3 | 0.26 | 3.13 |
| 7 | Compound 35 | 46.3:9.4:42.7:1.6 | 88.2 | 105.5 | 0.22 | −3.51 |
| 8 | Compound 40 | 46.3:9.4:42.7:1.6 | 90.1 | 101.2 | 0.20 | 3.65 |
| 9 | DLin-MC3-DMA** | 50:10:38.5:1.5 | 87.6 | 99.2 | 0.24 | 3.02 |

*DLin-MC3-DMA is a cationic lipid for the commercial nucleic acid delivery system Onpattro.

### Example 14—Determination of the Effect of Delivering Luciferase mRNA for In Vivo Expression Using Nano-Lipid Particle Compositions

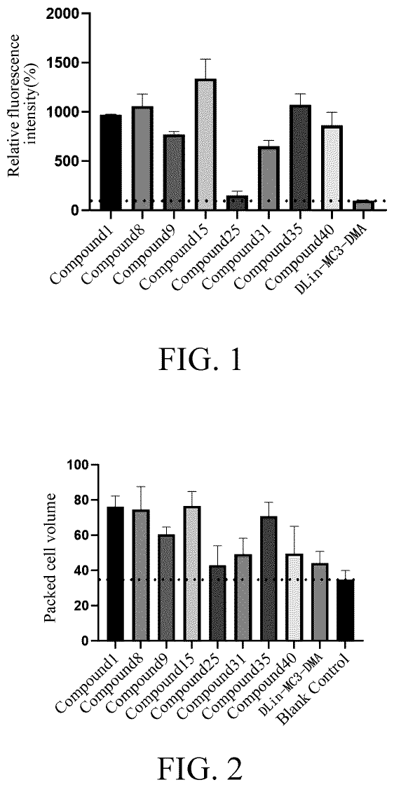

LUC-mRNA-lipid nanoparticles containing 3 ug mRNA (see SEQ ID NO:1 in the Patent Application No. 202210286081.0 for the corresponding nucleotide sequence of LUC-mRNA) were injected via tail intravenous injection in 6-8 week old female Balb/c mice, prepared as in Example 13. 200 ug of D-Luciferin Potassium Salt was intraperitoneally injected into the mice at specific time points and assayed using the Clinx Small Animal Imaging System. Fluc is commonly used in mammalian cell cultures to measure gene expression and cell viability. It emits biological light in the presence of the substrate fluorescein. The basic characteristics of the mRNA used include ARCA cap structure, polyA tail length of 100-120 nt, and complete substitution of pseudouridine. The results of the assay are shown in FIG. 1. The 1-8 #nano-lipid particle compositions delivered mRNA to the liver at levels superior to that of the MC3 nano-lipid particles.

### Example 15—Determination of the Expression and Effect of Delivering Erythropoietin (EPO) mRNA Using Nano-Lipid Particle Compositions in Mice

10 ug EPO-mRNA-lipid nanoparticles (see SEQ ID NO:2 in the Patent Application No. 202210286081.0 for the corresponding nucleotide sequence of EPO-mRNA) were injected via tail vein injection in 6-8 week old female Balb/c mice, prepared as in Example 13. At 48 hours after injection, eye frame blood sampling was performed on mice and the packed cell volume was determined. hEPO is commonly used as a characterization gene for the level of protein expression in mammalian blood, which is proportional to the packed cell volume. The basic characteristics of the mRNA used include ARCA cap structure, polyA tail length of 100-120 nt, and complete substitution of pseudouridine. The results of the assay are shown in FIG. 2. Seen from the results, multiple nano-lipid particle compositions comprising cations of the present patent delivered mRNA at levels superior to DLin-MC3-DMA.

### Example 16—In Vivo Metabolic Rates of Various Cationic Lipids

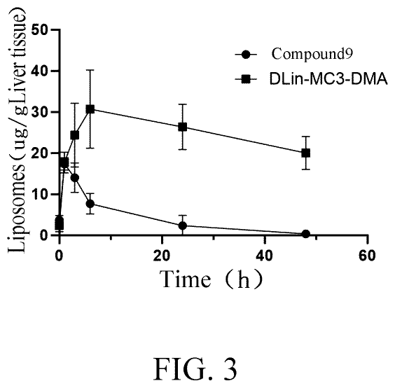

5 ug EPO-mRNA-lipid nanoparticles were injected via tail vein injection in 6-8 week old female Balb/c mice, prepared as in Example 13. After injection, the mice were executed at different time points (1 h, 3 h, 6 h, 24 h, 48 h after injection). The livers were taken and crushed, and the lipids were extracted from them with chloroform in three times, and the extracts were finally combined. After spin distillation to remove chloroform, methanol was added to dissolve the sample, and the cationic lipid content therein was analyzed by HPLC-CAD (Thermo Vanquish), with an Acclaim™ C18 column as the analytical column, a 0.5% TEAA aqueous solution as mobile phase A and a 0.5% TEAA methanol solution as mobile phase B. The sample was eluted according to the gradient in Table 2 below. The results are shown in FIG. 3.

TABLE 2

| No. | Time (min) | Flow rate (mL/min) | A(%) | B(%) |
|---|---|---|---|---|
| 1 | 0.00 | 0.4 | 15 | 85 |
| 2 | 5.00 | 0.4 | 10 | 90 |
| 3 | 15.00 | 0.4 | 0 | 100 |
| 4 | 50.00 | 0.4 | 0 | 100 |

From the results, it can be seen that compound 9 is metabolized in vivo faster than DLin-MC3-DMA, which means it has a better biosafety.

1. A cationic lipid compound having the structure of formula (I): 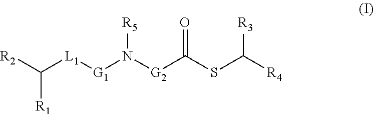
or pharmaceutically acceptable salts, tautomers or stereoisomers thereof, for delivery of nucleic acids,
wherein:
L₁ is any one of —C(═O)—, —OC(═O)—, —C(═O)O—, —OC(═O)O—, —O—, —S—, —S—S—, —C(═O)S—, —SC(═O)—, —N(R₆)C(═O)—, —C(═O)N(R₆)—, —N(R₆)C(═O)O—, —OC(═O)N(R₆)—, —SC(═O)N(R₆)—, —N(R₆)C(═O)S—, —C(═S)—, —SC(═S)— and —C(═S)S—, and R₆ is H or a C₁-C₁₂ alkyl;
G₁ and G₂ are each independently a C₃-C₁₀ alkylene;
R₁ is a C₂-C₁₂ alkyl;
R₂ is H or a C₂-C₁₂ alkyl;
R₃ is a C₂-C₁₂ alkyl;
R₄ is H or a C₂-C₁₂ alkyl;
R₅ is H, a C₁-C₆ alkyl, —R₇—OH, —R₇—OC(═O)CH₃, —R₇—NHC(═O)—CH₃, —R₇—OCH₃ or —R₇—NR₈(R₉);
R₇ is a C₂-C₁₈ alkylene;
R₈ and R₉ are C₁-C₈ straight chain alkyl groups, or R₈, R₉ and the N atom attached thereto form a C₃-C₁₀ heterocyclic group.

2. The cationic lipid compound according to claim 1, wherein L₁ is —OC(═O)—, —C(═O)O—, —SC(═O)— or —C(═O)S—.

3. The cationic lipid compound according to claim 2, wherein G₁ and G₂ are C₃-C₈ alkylene.

4. The cationic lipid compound according to claim 3, wherein R₇ is a C₂-C₆ alkylene.

5. The cationic lipid compound according to claim 4, wherein R₅ is —R₇—OH.

6. The cationic lipid compound according to claim 5, wherein R₁ and R₃ are each independently a C₃-C₉ alkyl; and R₂ and R₄ are H or C₃-C₉ alkyl.

7. The cationic lipid compound according to claim 5, wherein the structure of —C(R₁)R₂ or —C(R₃)R₄ in the structure of formula (I) each independently conforms to the following characteristics.

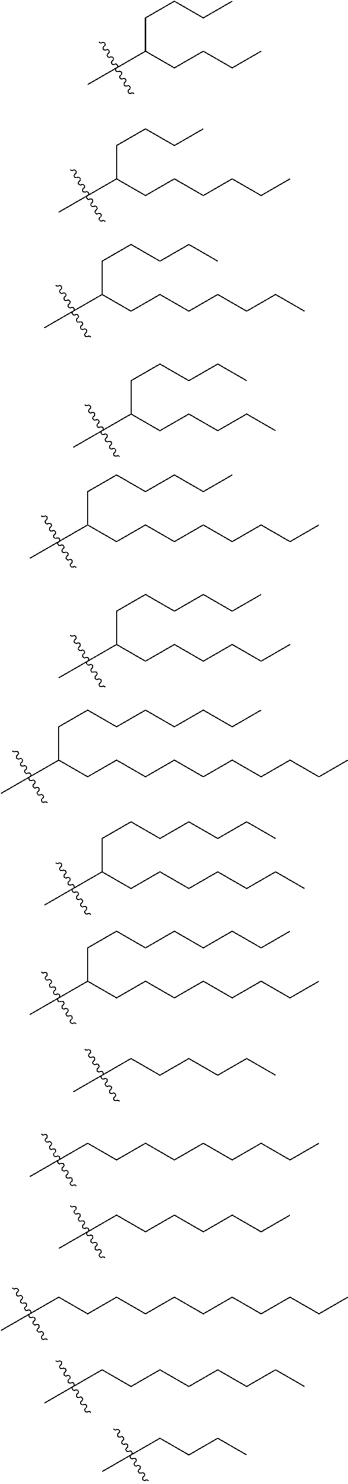

8. The cationic lipid compound according to claim 1, wherein the cationic lipid compound has one of the structures shown in the following table.

| No. | Structure |
|---|---|
| 1 | 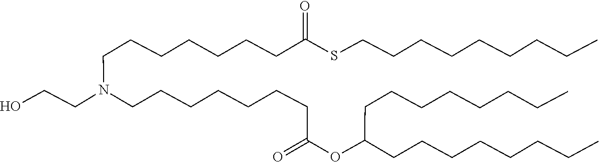 [SMILES: CCCCCCCCCSC(=O)CCCCCCCN(CCO)CCCCCCCC(=O)OC(CCCCCCCC)CCCCCCCC] |
| 2 | 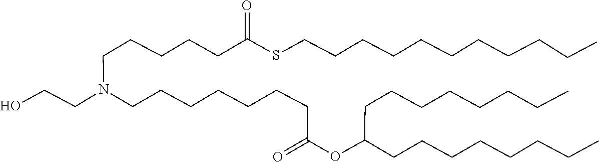 [SMILES: CCCCCCCCCCCSC(=O)CCCCCN(CCO)CCCCCCCC(=O)OC(CCCCCCCC)CCCCCCCC] |
| 3 | 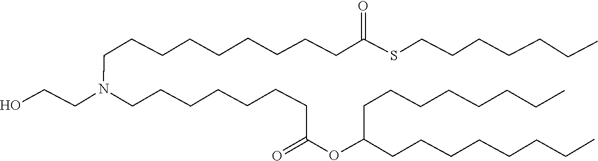 [SMILES: CCCCCCCCC(CCCCCCCC)OC(=O)CCCCCCCN(CCO)CCCCCCCCCC(=O)SCCCCCCC] |
| 4 | 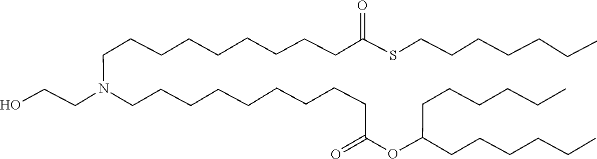 [SMILES: CCCCCCCSC(=O)CCCCCCCCCN(CCO)CCCCCCCCCC(=O)OC(CCCCCC)CCCCCC] |
| 5 | 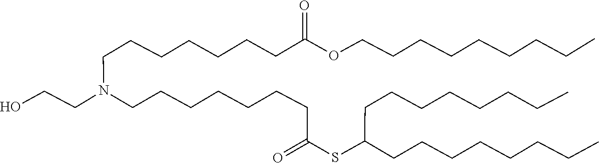 [SMILES: CCCCCCCCCOC(=O)CCCCCCCN(CCO)CCCCCCCC(=O)SC(CCCCCCCC)CCCCCCCC] |
| 6 | 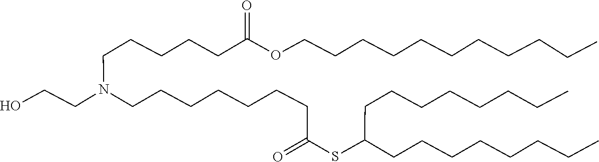 [SMILES: CCCCCCCCCCCOC(=O)CCCCCN(CCO)CCCCCCCC(=O)SC(CCCCCCCC)CCCCCCCC] |
| 7 | 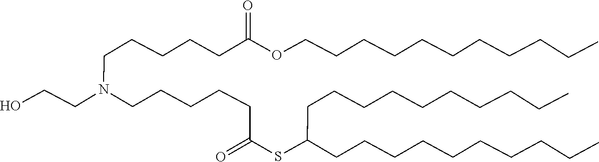 [SMILES: CCCCCCCCCCCOC(=O)CCCCCN(CCO)CCCCCC(=O)SC(CCCCCCCCCC)CCCCCCCCCC] |
| 8 | 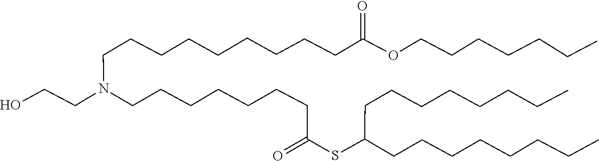 [SMILES: CCCCCCCCC(CCCCCCCC)SC(=O)CCCCCCCN(CCO)CCCCCCCCCC(=O)OCCCCCCC] |
| 9 | 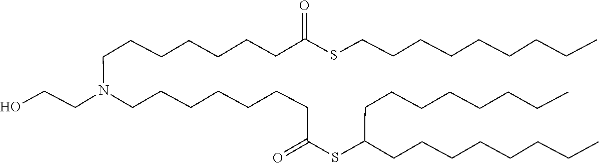 [SMILES: CCCCCCCCCSC(=O)CCCCCCCN(CCO)CCCCCCCC(=O)SC(CCCCCCCC)CCCCCCCC] |
| 10 | 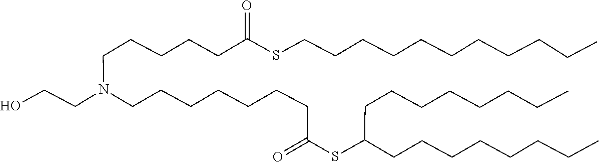 [SMILES: CCCCCCCCCCCSC(=O)CCCCCN(CCO)CCCCCCCC(=O)SC(CCCCCCCC)CCCCCCCC] |
| 11 | 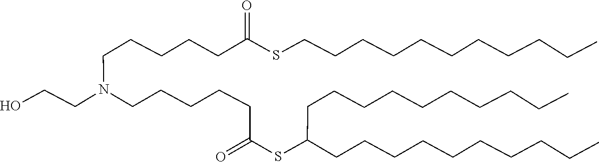 [SMILES: CCCCCCCCCCCSC(=O)CCCCCN(CCO)CCCCCC(=O)SC(CCCCCCCCCC)CCCCCCCCCC] |
| 12 | 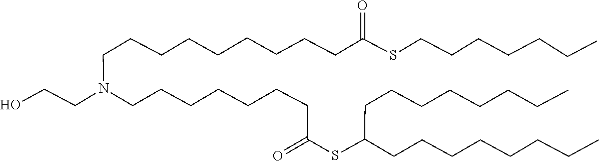 [SMILES: CCCCCCCCC(CCCCCCCC)SC(=O)CCCCCCCN(CCO)CCCCCCCCCC(=O)SCCCCCCC] |
| 13 | 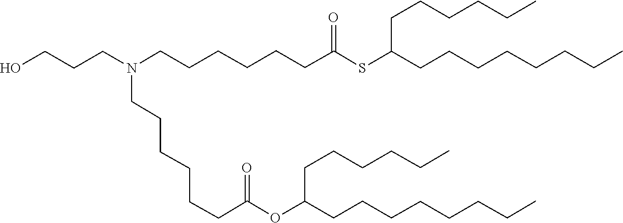 [SMILES: CCCCCCCCC(CCCCCC)OC(=O)CCCCCCN(CCCO)CCCCCCC(=O)SC(CCCCCC)CCCCCCCC] |
| 14 | 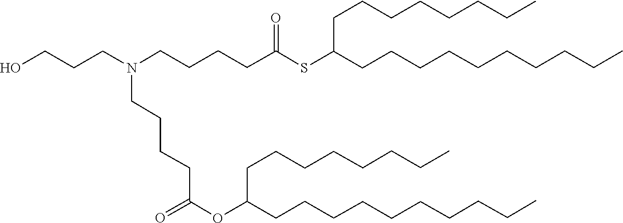 [SMILES: CCCCCCCCCCC(CCCCCCCC)OC(=O)CCCCN(CCCO)CCCCC(=O)SC(CCCCCCCC)CCCCCCCCCC] |
| 15 | 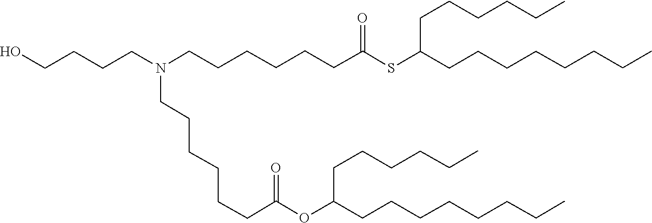 [SMILES: CCCCCCCCC(CCCCCC)OC(=O)CCCCCCN(CCCCO)CCCCCCC(=O)SC(CCCCCC)CCCCCCCC] |
| 16 | 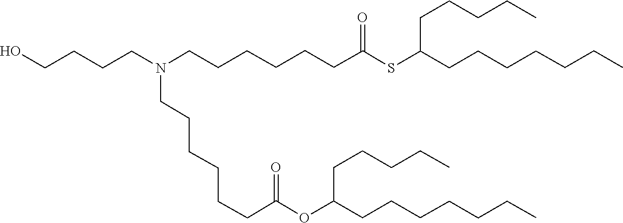 [SMILES: CCCCCCCC(CCCCC)OC(=O)CCCCCCN(CCCCO)CCCCCCC(=O)SC(CCCCC)CCCCCCC] |
| 17 | 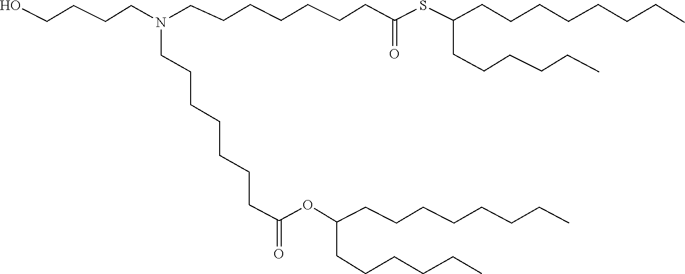 [SMILES: CCCCCCCCC(CCCCCC)OC(=O)CCCCCCCN(CCCCO)CCCCCCCC(=O)SC(CCCCCC)CCCCCCCC] |
| 18 | 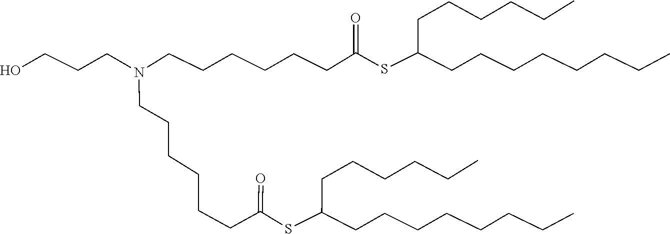 [SMILES: CCCCCCCCC(CCCCCC)SC(=O)CCCCCCN(CCCO)CCCCCCC(=O)SC(CCCCCC)CCCCCCCC] |
| 19 | 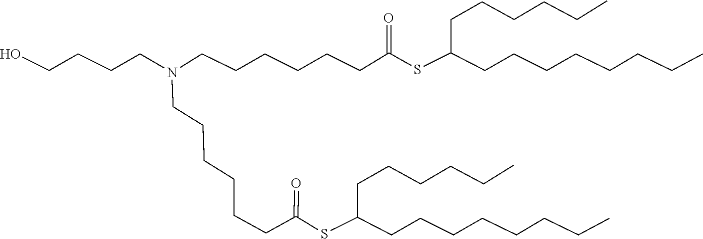 [SMILES: CCCCCCCCC(CCCCCC)SC(=O)CCCCCCN(CCCCO)CCCCCCC(=O)SC(CCCCCC)CCCCCCCC] |
| 20 | 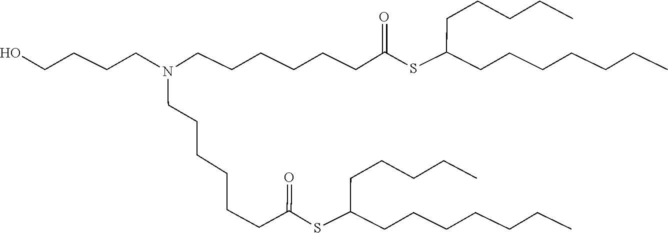 [SMILES: CCCCCCCC(CCCCC)SC(=O)CCCCCCN(CCCCO)CCCCCCC(=O)SC(CCCCC)CCCCCCC] |
| 21 | 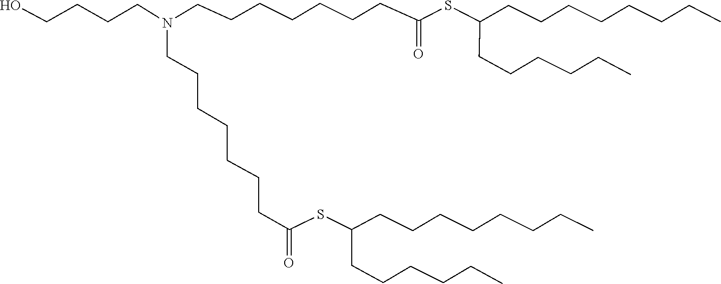 [SMILES: CCCCCCCCC(CCCCCC)SC(=O)CCCCCCCN(CCCCO)CCCCCCCC(=O)SC(CCCCCC)CCCCCCCC] |
| 22 | 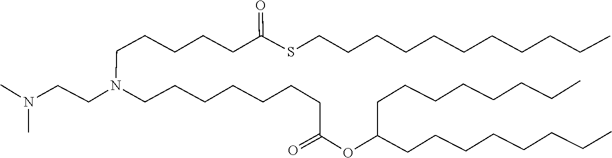 [SMILES: CCCCCCCCCCCSC(=O)CCCCCN(CCCCCCCC(=O)OC(CCCCCCCC)CCCCCCCC)CCN(C)C] |
| 23 | 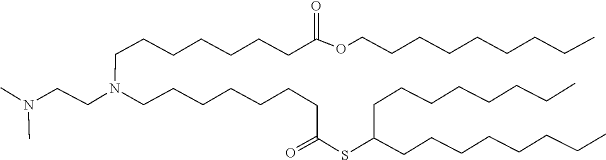 [SMILES: CCCCCCCCCOC(=O)CCCCCCCN(CCCCCCCC(=O)SC(CCCCCCCC)CCCCCCCC)CCN(C)C] |
| 24 | 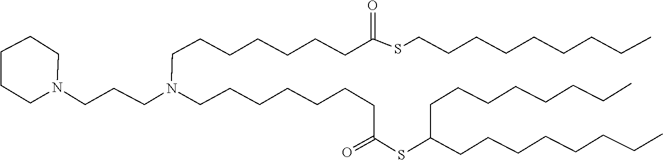 [SMILES: CCCCCCCCCSC(=O)CCCCCCCN(CCCCCCCC(=O)SC(CCCCCCCC)CCCCCCCC)CCCN1CCCCC1] |
| 25 | 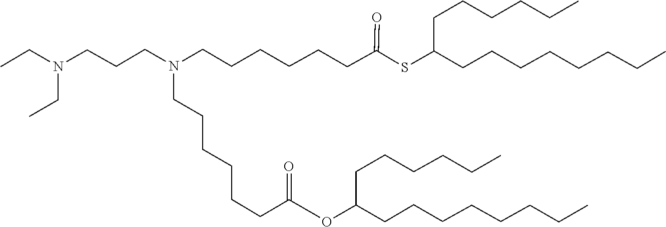 [SMILES: CCCCCCCCC(CCCCCC)OCC(=O)CCCCCCN(CCCCCCC(=O)SC(CCCCCC)CCCCCCCC)CCCN(CC)CC] |
| 26 | 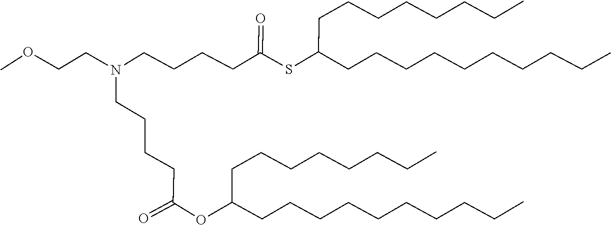 [SMILES: CCCCCCCCCCC(CCCCCCCC)OC(=O)CCCCN(CCCCC(=O)SC(CCCCCCCC)CCCCCCCCCC)CCOC] |
| 27 | 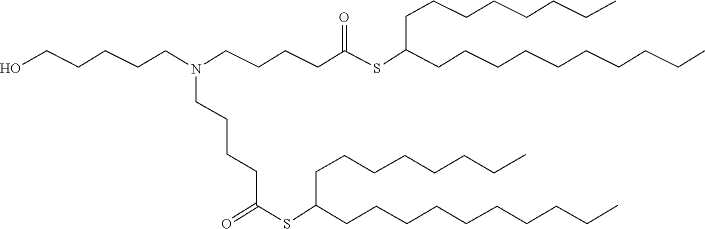 [SMILES: CCCCCCCCCCC(CCCCCCCC)SC(=O)CCCCN(CCCCCO)CCCCC(=O)SC(CCCCCCCC)CCCCCCCCCC] |
| 28 | 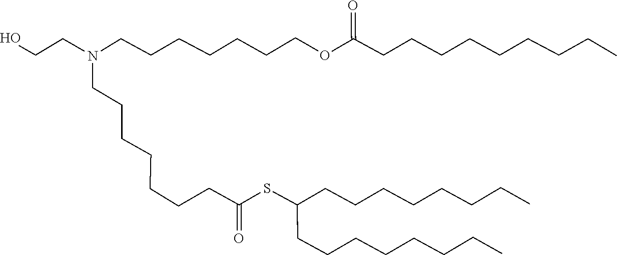 [SMILES: CCCCCCCCCC(=O)OCCCCCCCN(CCO)CCCCCCCC(=O)SC(CCCCCCCC)CCCCCCCC] |
| 29 | 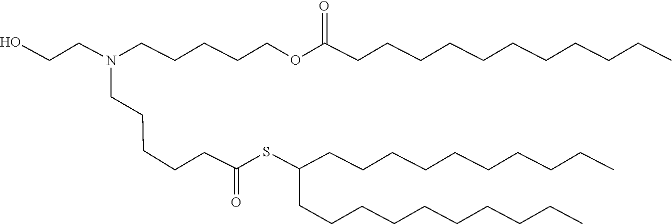 [SMILES: CCCCCCCCCCCC(=O)OCCCCCN(CCO)CCCCCC(=O)SC(CCCCCCCCCC)CCCCCCCCCC] |
| 30 | 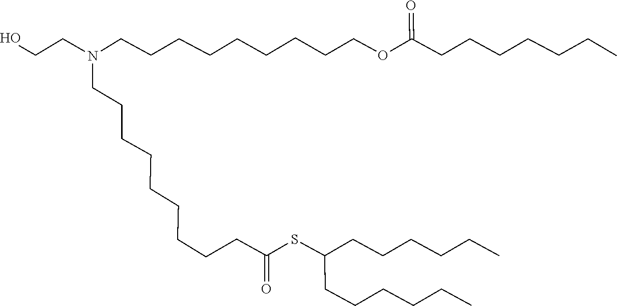 [SMILES: CCCCCCCC(=O)OCCCCCCCCCN(CCO)CCCCCCCCCC(=O)SC(CCCCCC)CCCCCC] |
| 31 | 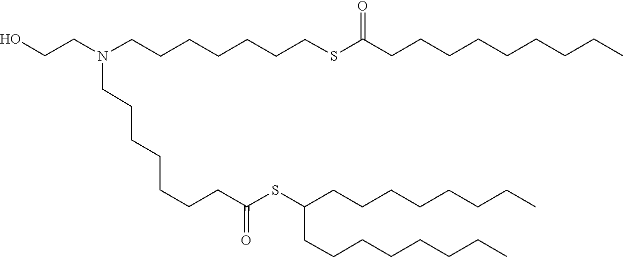 [SMILES: CCCCCCCCCC(=O)SCCCCCCCN(CCO)CCCCCCCC(=O)SC(CCCCCCCC)CCCCCCCC] |
| 32 | 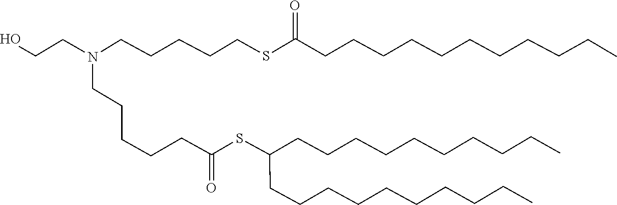 [SMILES: CCCCCCCCCCCC(=O)SCCCCCN(CCO)CCCCCC(=O)SC(CCCCCCCCCC)CCCCCCCCCC] |
| 33 | 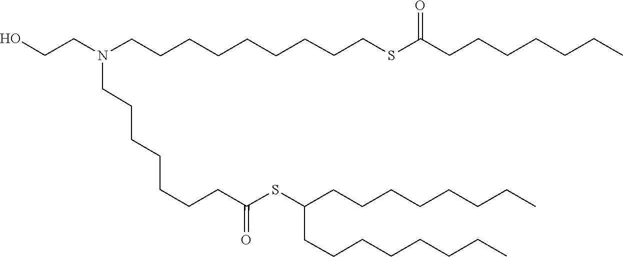 [SMILES: CCCCCCCCC(CCCCCCCC)SC(=O)CCCCCCCN(CCO)CCCCCCCCCSC(=O)CCCCCCC] |
| 34 | 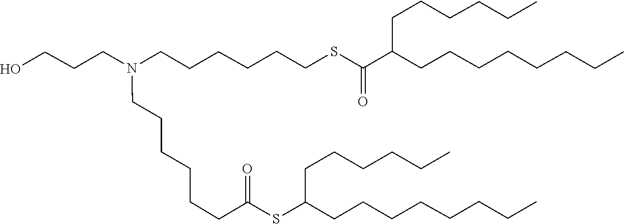 [SMILES: CCCCCCCCC(CCCCCC)SC(=O)CCCCCCN(CCCO)CCCCCCSC(=O)C(CCCCCC)CCCCCCCC] |
| 35 | 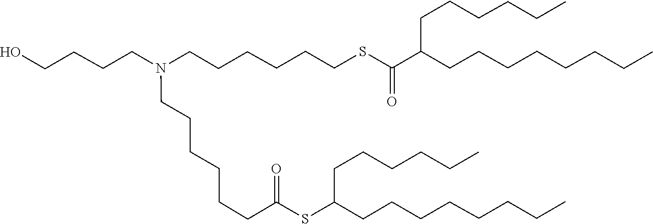 [SMILES: CCCCCCCCC(CCCCCC)SC(=O)CCCCCCN(CCCCO)CCCCCCSC(=O)C(CCCCCC)CCCCCCCC] |
| 36 | 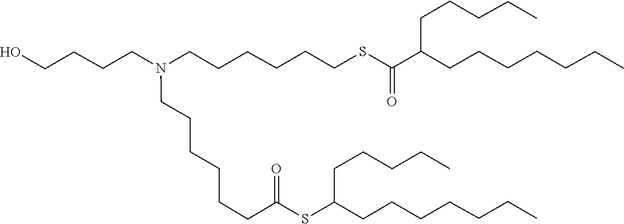 [SMILES: CCCCCCCC(CCCCC)SC(=O)CCCCCCN(CCCCO)CCCCCCSC(=O)C(CCCCC)CCCCCCC] |
| 37 | 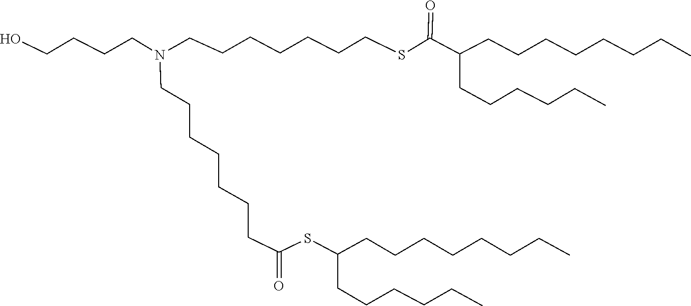 [SMILES: CCCCCCCCC(CCCCCC)SC(=O)CCCCCCCN(CCCCO)CCCCCCCSC(=O)C(CCCCCC)CCCCCCCC] |
| 38 | 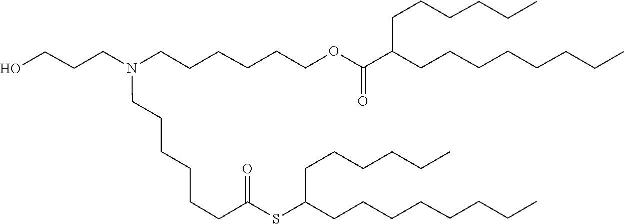 [SMILES: CCCCCCCCC(CCCCCC)SC(=O)CCCCCCN(CCCO)CCCCCCOC(=O)C(CCCCCC)CCCCCCCC] |
| 39 | 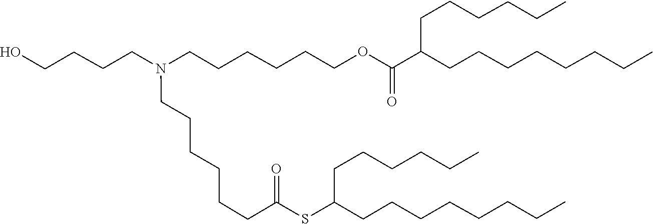 [SMILES: CCCCCCCCC(CCCCCC)SC(=O)CCCCCCN(CCCCO)CCCCCCOC(=O)C(CCCCCC)CCCCCCCC] |
| 40 | 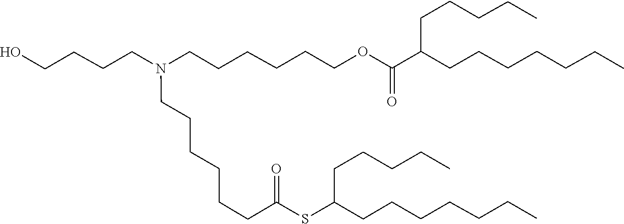 [SMILES: CCCCCCCC(CCCCC)SC(=O)CCCCCCN(CCCCO)CCCCCCOC(=O)C(CCCCC)CCCCCCC] |
| 41 | 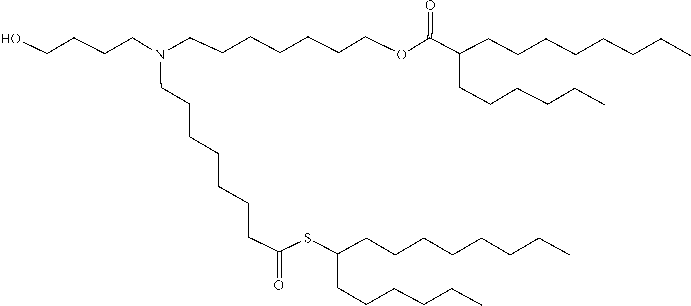 [SMILES: CCCCCCCCC(CCCCCC)SC(=O)CCCCCCCN(CCCCO)CCCCCCCOC(=O)C(CCCCCC)CCCCCCCC] |
| 42 | 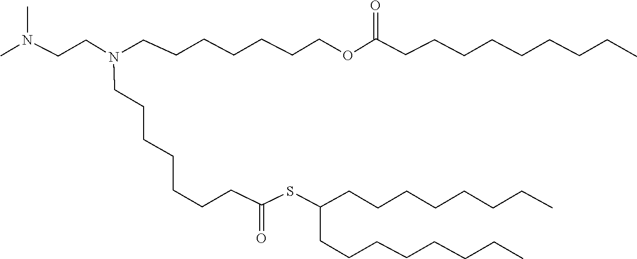 [SMILES: CCCCCCCCCC(=O)OCCCCCCCN(CCCCCCCC(=O)SC(CCCCCCCC)CCCCCCCC)CCN(C)C] |
| 43 | 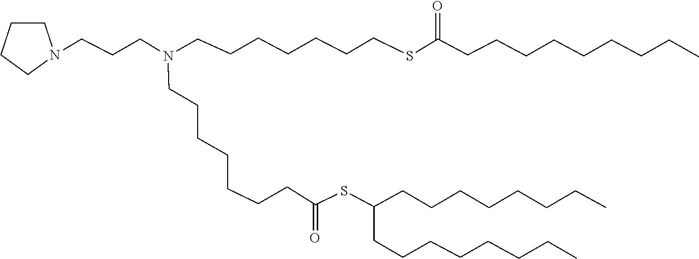 [SMILES: CCCCCCCCCC(=O)SCCCCCCCN(CCCCCCCC(=O)SC(CCCCCCCC)CCCCCCCC)CCCN1CCCC1] |
| 44 | 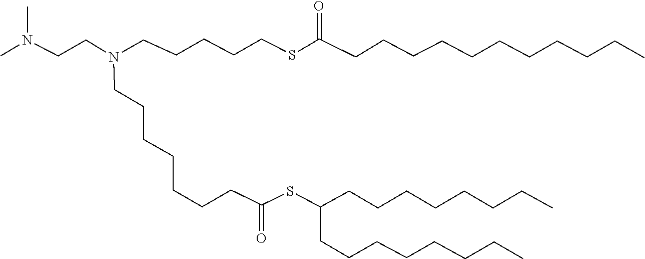 [SMILES: CCCCCCCCCCCC(=O)SCCCCCN(CCCCCCCC(=O)SC(CCCCCCCC)CCCCCCCC)CCN(C)C] |
| 45 | 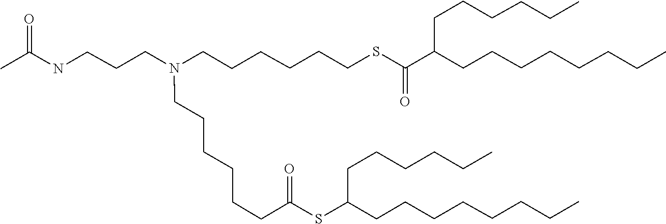 [SMILES: CCCCCCCCC(CCCCCC)SC(=O)CCCCCCN(CCCCCCSC(=O)C(CCCCCC)CCCCCCCC)CCCNC(C)=O] |
| 46 | 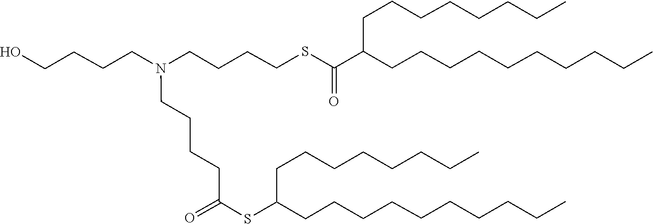 [SMILES: CCCCCCCCCCC(CCCCCCCC)SC(=O)CCCCN(CCCCO)CCCCSC(=O)C(CCCCCCCC)CCCCCCCCCC] |

9. A liposomal formulation comprising the cationic lipid compound according to claim 1 and a prophylactic or therapeutic nucleic acid, wherein the liposomal formulation is used for the prevention or treatment of diseases.

10. The liposomal formulation according to claim 9, wherein the molar ratio of the nucleic acid to the cationic lipid compound is from 20:1 to 1:1.

11. The liposomal formulation according to claim 9, wherein the liposomal formulation has a diameter of 50 nm to 300 nm.

12. The liposomal formulation according to claim 9, further comprising one or more other lipid components, including a structural lipid, a steroid and a polymer-conjugated lipid.

13. The liposomal formulation according to claim 12, wherein the included steroid is cholesterol.

14. The liposomal formulation according to claim 13, wherein the molar ratio of the cholesterol to the cationic lipid compound is (0-1.5):1.

15. The liposomal formulation according to claim 12, wherein the polymer in the polymer-conjugated lipid is polyethylene glycol (PEG).

16. The liposomal formulation according to claim 15, wherein the molar ratio of the compound to the polyethylene glycol-conjugated lipid is from 100:1 to 20:1.

17. The liposomal formulation according to claim 12, wherein the structural lipid is one or more selected from DPPG, DSPC, DPPC, DMPC, DOPC, POPC, DOPE and DSPE.

18. The liposomal formulation according to claim 17, wherein the molar ratio of the structural lipid to the cationic lipid compound is (0-0.5):1.

19. The liposomal formulation according to claim 9, wherein the nucleic acid is selected from antisense RNA and/or messenger RNA.

20. A method for inducing protein expression in a subject, wherein, the method comprises administering the cationic lipid compound according to claim 1 to a subject.
